(19) United States
(12) Patent Application Publication
(10) Pub. No.: US 2024/0263186 A1
(43) Pub. Date: Aug. 8, 2024

(54) EXPRESSION CONSTRUCTS, VIRD2 MUTANT AGROBACTERIUM STRAINS, AND METHODS OF USE THEREOF

(71) Applicant: PURDUE RESEARCH FOUNDATION, West Lafayette, IN (US)

(72) Inventors: Stanton B. GELVIN, West Lafayette, IN (US); Lan-Ying LEE, West Lafayette, IN (US)

(21) Appl. No.: 18/632,189

(22) Filed: Apr. 10, 2024

Related U.S. Application Data
(63) Continuation-in-part of application No. 17/494,977, filed on Oct. 6, 2021.
(60) Provisional application No. 63/533,332, filed on Aug. 17, 2023.
(60) Provisional application No. 63/458,275, filed on Apr. 10, 2023.
(60) Provisional application No. 63/089,567, filed on Oct. 9, 2020.

(51) Int. Cl. C12N 15/82; C07K 14/195; C12N 15/74

(52) U.S. Cl. CPC C12N 15/8205; C07K 14/195; C12N 15/743

(57) ABSTRACT

Agrobacterium strains with mutations in the VirD2 gene and protein and expression constructs and genome-editing systems for leveraging the same. Methods are also provided for Agrobacterium-mediated incorporation of exogenous expressible nucleic acids into a host plant material using the Agrobacterium strains, expression constructs, and/or genome-editing systems.

### PRIORITY

This patent application is related to, claims the priority benefit of: (1) and is a continuation-in-part of U.S. patent application Ser. No. 17/494,977 filed Oct. 6, 2021, and which claims priority to U.S. Provisional Patent Application 63/089,567 filed Oct. 9, 2020; (2) U.S. Provisional Patent Application No. 63/458,275 filed Apr. 10, 2023; and (3) U.S. Provisional Patent Application No. 63/533,332 filed Aug. 17, 2023. The contents of the foregoing applications are hereby incorporated by reference in their entireties into this disclosure.

### GOVERNMENT SUPPORT

This invention was made with government support under IOS1725122 awarded by the National Science Foundation. The government has certain rights in the invention.

### TECHNICAL FIELD

The present disclosure relates generally to Agrobacterium species for transforming a plant and, more particularly, to Agrobacterium species that transfer T (transfer)-deoxyribonucleic acid (DNA) into a plant without integrating the T-DNA into the genome of such plant.

### SEQUENCE LISTINGS

The sequences herein (SEQ ID NOS: 1-34) are also provided in computer readable form encoded in a file filed herewith and incorporated herein by reference, which was created on Apr. 10, 2024, named 69237-03_SequenceListing_10APR2024.xml, and is 49,152 bytes in size. The information recorded in computer readable form is identical to the written Sequence Listings provided below, pursuant to 37 C.F.R. § 1.821(f).

### BACKGROUND

This section introduces aspects that may help facilitate a better understanding of the disclosure. Accordingly, these statements are to be read in this light and are not to be understood as admissions about what is or is not prior art.

Agrobacterium species genetically transform plants by transferring a region of a plasmid (e.g., a tumor inducing (Ti)-plasmid) to plants. This region is called T (transfer)-deoxyribonucleic acid (DNA). T-DNA is initially processed from the Ti-plasmid by the activity of two proteins, VirD1 and VirD2. These two virulence (Vir) proteins form a complex and nick the T-DNA region of the Ti-plasmid within the T-DNA border repeat regions, which are 25 base pairs (bp) near-identical sequences that flank and delimit the T-DNA region.

During this nicking process, VirD2 protein covalent attaches to the 5′ end of one of the T-DNA strands (T-strands) through a phosphotyrosine linkage. The VirD2/T-strand complex subsequently is peeled off from the Ti-plasmid, forming a complex of VirD2 attached to the single-strand T-strand. It is this complex that is transferred from Agrobacterium to plant cells. Once in the plant, the T-strand DNA is thought to be coated by VirE2 protein, another Virulence protein made by Agrobacterium and transferred to the plant, to form a T-complex.

VirD2 is thought to be the protein most responsible for targeting T-strands to the plant nucleus. Once inside the nucleus, T-strands (either before or after replication to a double-strand DNA form; the mechanism is still unknown) are integrated into the plant genome, generating a stably transformed transgenic plant. The role of VirD2 in T-DNA integration into the plant genome is still not clear. However, it is thought that VirD2 does play a role. VirD2 has numerous protein domains, including a tyrosine²⁹ through which VirD2 covalently links to T-DNA, an N-terminal relaxase domain, a central domain of unknown function (DUF), a bipartite C-terminal nuclear localization signal (NLS) sequence, and a small domain near the C-terminus of the protein called ω. Alteration of ω by deletion of four amino acids and substitution of two serine residues (the ω mutation) results in an Agrobacterium strain that transfer T-DNA—4-5 fold less efficiently (transient transformation), but which integrates T-DNA-50-fold less efficiently (stable transformation). Thus the ω mutation results in an Agrobacterium strain that partially uncouples T-DNA transfer from T-DNA integration.

Previously, scientists generated genome-edited plants by transferring T-DNA containing genome editing reagents (such as clustered regularly interspaced short palindromic repeats (CRISPR)-Cas) and having this T-DNA integrate into the plant genome. After the genome engineering reagents functioned to create the desired mutations, the plant chromosome containing the T-DNA was eliminated by genetic crossing to generate an edited, but non-transgenic organism. However, genetic crossing is often not practical or economically feasible for plants (such as trees) with long generation times, or for plants (such as banana, potato, or sweet potato, for example) that are normally vegetatively propagated.

Accordingly, there is a need for new efficient and effective tools for genetic engineering of plant cells and plants. For genome engineering purposes, it would be advantageous to generate an Agrobacterium strain that can efficiently transfer T-DNA but not integrate it. That way, one could deliver genome engineering reagents that can edit the genome, but not integrate T-DNA containing these reagents such that the resulting plant is not transgenic, which is subject to regulation in many countries.

In view of the above, it is an object of the present disclosure to provide useful and novel recombinant DNA constructs, VirD2 mutant genes and Agrobacterium strains that allow for the genomic engineering of plant cells. Certain methods and approaches to generate such mutants have been described, as in U.S. Patent Application Publication No. 2022/0112510 A1, which is hereby incorporated by reference in its entirety. This and other objects and advantages, as well as inventive features, will be apparent from the detailed description provided herein.

### SUMMARY

Agrobacterium mutant strains are provided. In certain embodiments, an Agrobacterium strain comprises a mutant VirD2 gene that effects transient transformation of a plant, the mutant VirD2 strain encoding a wild-type VirD2 protein except for:
(i) a mutation at a threonine at position 319 of the VirD2 protein;
(ii) a mutation at a threonine at position 319 of the VirD2 protein, a mutation at a histidine at position 402 of the VirD2 protein, and an insertion of 30 to 40 amino acid residues at a terminal end of the VirD2 protein resulting from a truncation mutation at a glutamine at position 425; or
(iii) an insertion of 1 to 20 amino acid residues after position 326 of the VirD2 protein, which affects a downstream frameshift.

The wild-type VirD2 protein can be or comprise SEQ ID NO: 16 or SEQ ID NO: 17. The mutant VirD2 gene can be preceded by an enhanced Shine-Dalgarno sequence such as SEQ ID NO: 31 or a functional variant of SEQ ID NO: 31.

In certain embodiments, the mutation at position 319 of the VirD2 protein comprises substitution of threonine with an amino acid other than threonine; the mutation at position 402 of the VirD2 protein comprises substitution of histidine with an amino acid other than histidine; and/or the truncation of position 425 of the VirD2 protein results from a mutation of a final stop codon encoded by a wild-type VirD2 gene (any combination of the foregoing). In certain embodiments, the mutation at position 319 of the VirD2 protein is a substitution of threonine with alanine. In certain embodiments, the mutation at position 402 of the VirD2 protein comprises substitution of histidine with arginine.

In certain embodiments, the insertion of 1 to 20 amino acid residues after position 326 of the VirD2 protein is or comprises SEQ ID NO: 30 or a functional variant thereof. In certain embodiments, the insertion of 1 to 20 amino acid residues after position 326 of the VirD2 protein is encoded by SEQ ID NO: 29 or a functional variant thereof. In certain embodiments, the insertion of 1 to 20 amino acid residues after position 326 of the VirD2 protein is or comprises SEQ ID NO: 30 or a functional variant thereof. In certain embodiments, the insertion of 1 to 20 amino acid residues after position 326 of the VirD2 protein is encoded by SEQ ID NO: 29 or a functional variant thereof.

In certain embodiments, the insertion of 30 to 40 amino acid residues is or comprises SEQ ID NO: 32 or a functional variant thereof.

The Agrobacterium strain can have at least less stable transformation of a plant than an Agrobacterium comprising a wild-type VirD2 gene or no stable transformation of a plant.

The VirD2 protein can be or comprise SEQ ID NO: 19, SEQ ID NO: 23, SEQ ID NO: 25, or a functional variant of SEQ ID NO: 19, SEQ ID NO: 23, or SEQ ID NO: 25. The mutant VirD2 gene can be or comprise SEQ ID NO: 20, SEQ ID NO: 24, SEQ ID NO: 26, or a functional variant of SEQ ID NO: 20, SEQ ID NO: 24, or SEQ ID NO: 26.

The Agrobacterium strains hereof can be incorporated into an expression construct. The expression construct can comprise a nucleic acid construct and, optionally, a plasmid or a replicating plasmid. In certain embodiments, the expression construct comprises a root-inducing plasmid (Ri-plasmid) or a tumor-inducing plasmid (Ti-plasmid). The Ti-plasmid can be a pTiEHA105 plasmid. The wild-type VirD2 gene of the expression construct can be inactive, deleted, disrupted, disarmed, and/or replaced by the mutant VirD2 gene.

Expression constructs are also provided. A expression construct can comprise, for example, a mutant VirD2 gene that encodes a wild-type Agrobacterium VirD2 protein except for:
(i) a mutation at a threonine at position 319 of the VirD2 protein;
(ii) a mutation at a threonine at position 319 of the VirD2 protein, a mutation at a histidine at position 402 of the VirD2 protein, and an insertion of 30 to 40 amino acid residues at a terminal end of the VirD2 protein due to a truncation mutation at a glutamine at position 425; or
(iii) an insertion of 1 to 22 amino acid residues after position 326 of the VirD2 protein, which affects a downstream frameshift.

The expression construct can comprise a nucleic acid construct such as a plasmid or a replicating plasmid. In certain embodiments, for example where the expression construct comprises a nucleic acid construction, the expression construct comprises an Ri-plasmid or a Ti-plasmid. The Ti-plasmid can be a pTiEHA105 plasmid.

The wild-type VirD2 gene of the expression construct can be inactive, deleted, disrupted, and/or replaced by the mutant VirD2 gene. In certain embodiments, the wild-type VirD2 protein encoded by the expression construct is or comprises SEQ ID NO: 16 or SEQ ID NO: 17.

In certain embodiments, the mutant VirD2 gene can be preceded by an enhanced Shine-Dalgarno sequence such as SEQ ID NO: 31 or a functional variant of SEQ ID NO: 31. In certain embodiments of the expression construct, the VirD2 protein is or comprises SEQ ID NO: 19, SEQ ID NO: 23, SEQ ID NO: 25, or a functional variant of SEQ ID NO: 19, SEQ ID NO: 23, or SEQ ID NO: 25; or the mutant VirD2 gene is or comprises SEQ ID NO: 20, SEQ ID NO: 24, SEQ ID NO: 26, or a functional variant of SEQ ID NO: 20, SEQ ID NO: 24, or SEQ ID NO: 26.

In certain embodiments of the expression construct, the mutation at position 319 of the VirD2 protein comprises substitution of threonine with an amino acid other than threonine; the mutation at position 402 of the VirD2 protein comprises substitution of histidine with an amino acid other than histidine; and/or an insertion of 30 to 40 amino acid residues at a terminal end of the VirD2 protein comprises insertion of 38 amino acid residues. In certain embodiments, the insertion after position 326 of the VirD2 protein is 18 amino acid residues and, optionally, is or comprises SEQ ID NO: 30 or a functional variant thereof and/or is encoded by SEQ ID NO: 29 or a functional variant thereof.

The truncation of position 425 of the VirD2 protein can be or comprise SEQ ID NO: 32 or a functional variant thereof and can, for example, result from a mutation of a final stop codon encoded by a wild-type VirD2 gene.

The expression construct can further comprise one or more operating regulatory segments. Still further, the expression construct can further comprise, for example, where provided as part of a gene-editing system, a sequence-specific nuclease, a DNA polymerase, and/or a DNA polymerase recruitment protein, or an expression construct comprising a nucleotide sequence encoding the sequence-specific nuclease, the DNA polymerase and/or the DNA polymerase recruitment protein.

Genome editing systems (e.g., for a plant genome) are also provided, for example, that leverage the Agrobacterium strains and expression constructs hereof. In certain embodiments, the genome editing system comprises an expression construct comprising a mutant Agrobacterium VirD2 gene that encodes an Agrobacterium wild-type VirD2 protein except for:
(i) a mutation at a threonine at position 319 of the VirD2 protein,
(ii) a mutation at a threonine at position 319 of the VirD2 protein, a mutation at a histidine at position 402 of the VirD2 protein, and an insertion of 30 to 40 amino acid residues at a terminal end of the VirD2 protein resulting from a truncation of a glutamine at position 425 of the VirD2 protein, or
(iii) an insertion of 1 to 20 amino acid residues after position 326 of the VirD2 protein, which affects a downstream frameshift; and
a sequence-specific nuclease, a DNA polymerase, and/or a DNA polymerase recruitment protein, or an expression construct comprising a nucleotide sequence encoding the sequence-specific nuclease, the DNA polymerase and/or the DNA polymerase recruitment protein. The expression construct of the genome editing system can comprise any of the expression constructs described herein.

The sequence specific nuclease of the genome editing system can be a CRISPR nuclease, such as a CRISPR nickase, for example. The CRISPR nickase can be a Cas9 nickase. The expression construct can comprise a nucleotide sequence encoding the sequence specific nuclease comprises a T-DNA binary vector and the sequence specific nuclease is a Cas9 nickase.

The genome editing system can further comprise a guide RNA and/or an expression construct comprising a nucleotide sequence encoding the guide RNA. In certain embodiments, the genome editing system further comprises a guide RNA targeting a PDS2 gene of Nicotiana benthamiana. In certain embodiments, the genome editing system further comprises a guide RNA protospacer targeting a PDS2 gene of N. benthamiana and having or comprising SEQ ID NO: 12.

The mutant VirD2 gene of the expression construct of the genome editing system can be or comprise SEQ ID NO: 26 or a functional variant of SEQ ID NO: 26. In such embodiments, the gene editing system, in use, can affect CRISPR mutagenesis about 50% to about 80% as well as compared to a genome editing system employing a wild-type Agrobacterium VirD2 strain, for example.

Methods for Agrobacterium-mediated incorporation of exogenous expressible nucleic acids into a host plant material are also provided. In certain embodiments, the method comprises infecting a target host plant material with any of the mutant Agrobacterium strains provided herein. In certain embodiments, the method comprises infecting a target host plant material with an Agrobacterium strain comprising a mutant VirD2 gene that effects transient transformation of a plant, the mutant VirD2 strain encoding a wild-type VirD2 protein except for:
(i) a mutation at a threonine at position 319 of the VirD2 protein;
(ii) a mutation at a threonine at position 319 of the VirD2 protein, a mutation at a histidine at position 402 of the VirD2 protein, and an insertion of 30 to 40 amino acid residues at a terminal end of the VirD2 protein resulting from a truncation mutation at a glutamine at position 425; or
(iii) an insertion of 1 to 20 amino acid residues after position 326 of the VirD2 protein, which affects a downstream frameshift. By way of non-limiting example, the plant material can be selected from the group consisting of plant cells, leaves, roots, stems, buds, flowers, fruits, seeds, germinated seeds or plant tissues of any other parts, or whole plants.

Infecting can comprise, for example, inoculating the target host plant material with the Agrobacterium strain at a dose of at or between about 10⁶ cfu/ml to about 10⁹ cfu/ml. In certain embodiments, the dose is at or about 10⁸ cfu/ml.

In certain embodiments, the Agrobacterium strain of the method comprises a mutant VirD2 gene that is or comprises SEQ ID NO: 22 or a functional variant of SEQ ID NO: 22, and the dose is at or between about 10⁷ cfu/ml to about 10⁸ cfu/ml. In certain embodiments, the Agrobacterium strain of the method comprises a mutant VirD2 gene that is or comprises SEQ ID NO: 20, SEQ ID NO: 24, SEQ ID NO: 26, or a functional variant of SEQ ID NO: 20, SEQ ID NO: 24, or SEQ ID NO: 26, and the dose is at or between about 10⁷ cfu/ml to about 10⁸ cfu/ml.

The Agrobacterium strain of the method can comprise, for example, a mutant VirD2 gene that is or comprises SEQ ID NO: 26 or a functional variant of SEQ ID NO: 26 and have at least 80- to 100-fold less stable transformation as compared to an Agrobacterium comprising a wild-type VirD2 gene or no stable transformation. In certain embodiments of the method, the Agrobacterium strain comprises a mutant VirD2 gene that is or comprises SEQ ID NO: 26 or a functional variant of SEQ ID NO: 26, and transiently transforms the host plant material about 30-70% as well as compared to an Agrobacterium wild-type strain.

Plant cells that have been infected according to the methods hereof are also provided. Still further, a plant comprising a plant material which has been infected according to the methods hereof is provided. Use of the Agrobacterium strains hereof is also provided, as well as use of the expression constructs and/or the genome editing system hereof, to transiently transform a host plant cell of a plant such that the plant expresses one or more traits of interest.

### DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1 is a schematic diagram of the generation of VirD2/T-strand complexes in Agrobacterium. VirD2 nicks the transfer DNA (T-DNA) region of the tumor inducing (Ti)-plasmid within the border repeat sequences. VirD2 covalently links to the T-strand, forming a VirD2/T-strand complex. After transport to the plant cell, the single-strand T-strands are thought to be coated by VirE2 protein, which is also transported from Agrobacterium into the plant cell.

FIGS. 2A-2C illustrate the role of the VirD2 ω region in transient and stable transformation. FIG. 2A is a schematic diagram of the VirD2 protein, showing the various domains. *NLS, sequence that likely does not have nuclear localization signal (NLS) activity in vivo. Note the amino acid sequence in the C-terminus of the protein, including the active NLS and the ω domain. The amino acid sequence of wild-type and mutant ω domains are shown in black and gray, respectively. FIG. 2A discloses SEQ ID NOS: 13 and 14, respectively in order of appearance. FIG. 2B is images illustrating transient transformation of tobacco leaf discs by an Agrobacterium strain containing wild-type or ω mutant VirD2 proteins. Note that a strain with the ω mutation shows ˜5-fold less transient transformation (Escherichia coli β-glucuronidase gene uidA (GUS) activity) than does a strain with a wild-type VirD2. FIG. 2C is images illustrating stable transformation of Arabidopsis root segments by an Agrobacterium strain containing wild-type or ω mutant VirD2 proteins. Note that a strain with the ω mutation shows ˜50-fold less stable transformation (crown gall tumorigenesis) than does a strain with a wild-type VirD2.

FIG. 3 shows schematics of the VirD operon on a Ti-plasmid, with the upper map showing a VirD operon on the Ti-plasmid, with the various VirD genes and non-polar deletion in VirD2 shown, the middle map showing a blow-up of the promoter (P_{virD}) gene region (VirDₚᵣₒₘₒₜₑᵣ-virD1-virD2), and the lower map showing a schematic of cloned region of a VirD operon on a replicating plasmid in Agrobacterium with various random mutations generated in VirD2, including a mutagenized VirD2 region that was subjected to mutagenic polymerase chain reaction (PCR) pursuant to the methods hereof.

FIG. 4A shows an amino acid sequence (SEQ ID NO: 16) and a nucleotide sequence (SEQ ID NO: 15) of wild-type VirD2 (pE45e4, pE4770 indicating Gelvin laboratory strain numbers). Note the bipartite NLS is identified in a slashed box and the omega domain is identified in a dotted-line box. Tyrosine-29 links VirD2 to T-DNA

FIG. 4B shows an amino acid sequence (SEQ ID NO: 17) and a nucleotide sequence (SEQ ID NO: 18) of wild-type VirD2 (pE4896) with Shine-Dalgarno sequence and NcoI site. Note the bipartite NLS is identified in a slashed box and the omega domain is identified in a dotted-line box.

FIG. 4C shows an amino acid sequence (SEQ ID NO: 19) and a nucleotide sequence (SEQ ID NO: 20) VirD2 Mutant 1 (pE4896_4F03=pE4928), which is hypervirulent.

FIG. 4D shows an amino acid sequence (SEQ ID NO: 21) and a nucleotide sequence (SEQ ID NO: 22) of VirD2 Mutant 2 (pE4896_4E12).

FIG. 4E shows an amino acid sequence (SEQ ID NO: 23) and a nucleotide sequence (SEQ ID NO: 24) of VirD2 Mutant 3 (pE4896_4E12+4F03=pE4905) (“Double Mutant”).

FIG. 4F shows an amino acid sequence (SEQ ID NO: 25) and a nucleotide sequence (SEQ ID NO: 26) of VirD2 Mutant 4 (pE4896_w34A2/truncated+_extra=pE4960) (“T-mutant”).

FIG. 5 is a schematic diagram of building the Agrobacterium strains containing mutant VirD2 genes. A plasmid containing the VirDₚᵣₒₘₒₜₑᵣ-virD1-virD2ₘᵤₜₐₙₜ is introduced into an Agrobacterium strain containing a Ti-plasmid with a non-polar deletion of VirD2. This figure shows that the bacterium also contains the T-DNA binary vector pBISN1, but any compatible binary vector can be used. pBISN1 contains in its T-DNA a gusA-intron gene (to monitor stable transformation by GUS activity) and a Pnos-nptII gene (to monitor stable transformation by kanamycin resistance). The strain pictured also contains T-DNA that contains oncogenes such that the bacterium can incite tumors (stable transformation).

FIG. 6 shows an image of Kalanchoe tumorigenesis assays identified VirD2 mutants that did not affect stable transformation. Leaves of Kalanchoe diagremontiana were wounded, each wound was inoculated with a different Agrobacterium strain containing various VirD2 alleles, and the leaf was photographed after 1 month and the presence of tumors was scored. Note the positive (tumor) and negative (no tumor) controls. Of interest are virD2 mutant strains 5F09 and 4E12. These strains did not incite tumors, indicating a large decrease in stable transformation.

FIG. 7 shows images of X-gluc staining of tobacco leaves infiltrated with various virD2 mutant Agrobacterium strains. Tobacco leaves were infiltrated with various Agrobacterium strains and leaf sections from the infiltrated area were stained with X-gluc after 24 hr. At2201 is a positive control (wild-type virD2) and At2200 is a negative control (no virD2). Note that many virD2 mutant strains appear to transiently transform tobacco as well as does the wild-type virD2 strain.

FIG. 8. Quantitative stable transformation assay of Arabidopsis root segments infected with Agrobacterium strains harboring various mutant virD2 genes. Root segments from wild-type (ecotype Col-0) plants were infected with the various Agrobacterium strains at 10⁷ cfu/ml for two days. The roots were then moved to CIM medium containing timentin (to kill Agrobacterium) and kanamycin (to select for stable transformants). Kanamycin-resistant calli were scored after one month.

FIG. 9. Quantitative transient transformation assay of Arabidopsis root segments infected with Agrobacterium strains harboring various mutant virD2 genes. Root segments from wild-type (ecotype Col-0) plants were infected with the various Agrobacterium strains at 10⁸ cfu/ml for two days. The roots were then moved to CIM medium containing timentin (to kill Agrobacterium) and incubated for one month. Root segments were then stained with X-gluc for GUS activity. Note that mutant 4E12, which effects extremely low stable transformation activity, effects substantial transient transformation GUS activity.

FIG. 10. Mutation frequencies surrounding the Cas9 cleavage site in the Nicotiana benthamiana PDS2 gene. N. benthamiana leaves were infiltrated with an Agrobacterium strain containing a wild-type or mutant VirD2 gene. The T-DNA contained a Cas9 gene, a sgRNA gene directed against the tobacco PDS2 gene, and a Venus-intron gene. DNA was extracted from Venus-fluorescent tissue and a region surrounding the PDS2 cleavage site was amplified by PCR. The amplicons were sequenced via Wide-seq. Shown are the percentage of mutations at each site surrounding the Cas9 cleavage site. The number of reads (sequencing depth) at each nucleotide varied from ˜7000-27000. The percent mutations includes nucleotide substitutions, insertions, and deletions (most mutations are in/dels). Shown are the nucleotides surrounding the Cas9 cleavage site. The PAM (protospacer adjacent motif) sequence is also shown. All these VirD2 mutants show good transient GUS activity on infiltrated tobacco leaves, and extremely poor stable transformation of Kalanchoe and Arabidopsis. Note that these VirD2 mutants support editing around the Cas9 cleavage site at ˜25-40% the frequency of the wild-type VirD2 gene.

FIG. 11 shows an image of X-gluc staining of tobacco leaves infiltrated with various VirD2 mutant Agrobacterium strains. Nicotiana benthamiana leaves were infiltrated with various Agrobacterium strains harboring various VirD2 alleles for the indicated number of days. Leaf sections from the infiltrated area of each leaf were stained with X-gluc after 24 hours, then de-stained in 70% ethanol.

FIG. 12 is graphical data from a transient transformation assay of Arabidopsis root segments infected with Agrobacterium strains harboring wild-type or various mutant VirD2 genes. Root segments from wild-type (ecotype Col-0) plants (Wt) were infected with various Agrobacterium strains (VirD2 Mutant 1 (4F03), VirD2 Mutant 2 (4E12), and VirD2 Mutant 3 (Double Mutant; i.e., combined 4F03 and 4E12 mutations in the same VirD2 gene)) at different concentrations (K10, K1, or K0.1 indicates that the root segments were inoculated with 10⁸ cfu/ml, 10⁷ cfu/ml, or 10⁶ cfu/ml, respectively) for six (6) days. The Agrobacterium was then killed, and root segments were stained with X-gluc to assess GUS activity.

FIG. 13 is graphical data from a stable transformation assay of Arabidopsis root segments infected with Agrobacterium strains harboring wild-type or various mutant VirD2 genes. Root segments from wild-type (ecotype Col-0) plants (Wt) were infected with various Agrobacterium strains (VirD2 Mutant 1 (4F03), VirD2 Mutant 2 (4E12), and VirD2 Mutant 3 (Double Mutant; i.e., combined 4F03 and 4E12 mutations in the same VirD2 gene)) at different concentrations (K10, K1, or K0.1 indicates that the root segments were inoculated with 10⁸ cfu/ml, 10⁷ cfu/ml, or 10⁶ cfu/ml, respectively). One (1) month post-inoculation, root segments were assayed for tumor formation.

FIG. 14 is graphical data from a transient transformation assay of Arabidopsis root segments infected with Agrobacterium strains harboring wild-type or various mutant VirD2 genes. Root segments from wild-type (ecotype Col-0) plants (Wt) were infected with various Agrobacterium strains (Double Mutant=VirD2 Mutant 3, ω mutant, and T mutant=VirD2 Mutant 4) at different concentrations (K10, K1, or K0.1 indicates that the root segments were inoculated with 10⁸ cfu/ml, 10⁷ cfu/ml, or 10⁶ cfu/ml, respectively). The Agrobacterium was killed, and six (6) days post inoculation, root segments were stained with X-gluc to assess GUS activity. All mutant strains had about 50% of the wild-type activity for transient formation.

FIG. 15 is graphical data from a stable transformation assay of Arabidopsis root segments infected with Agrobacterium strains harboring wild-type or various mutant VirD2 genes. Root segments from wild-type (ecotype Col-0) plants (Wt) were infected with various Agrobacterium strains (Double Mutant=VirD2 Mutant 3, w mutant=, and T mutant=VirD2 Mutant 4) at different concentrations (K10 or K1 indicates that the root segments were inoculated with 10⁸ cfu/ml or 10⁷ cfu/ml, respectively). One (1) month post-inoculation, root segments were assayed for tumor formation. All mutant strains were extremely low for stable transformation activity.

FIG. 16 is a table of mutation frequency data surrounding the Cas9 cleavage site in the Nicotiana benthamiana PDS2 gene of N. Benthamiana leaves four (4) days post-infiltration with an Agrobacterium strain containing a wild-type (Wt) or mutant VirD2 gene (VirD2 Mutant 1 (4F03), VirD2 Mutant 2 (4E12), and VirD2 Mutant 3 (Double Mutant, i.e., combined 4F03 and 4E12 mutations in the same VirD2 gene)). The top row indicates the nucleotide number within the PDS2 amplicon and the numbers within the table are the percent mutations at each site surrounding the Cas9 cleavage site (between nucleotides 290 and 291 (bolded)). The percent mutations includes nucleotide substitutions, insertions, and deletions.

FIG. 17 is a table of mutation frequency data surrounding the Cas9 cleavage site in the PDS2 gene of N. Benthamiana leaves four (4) days post-infiltration with an Agrobacterium strain containing a wild-type (Wt) or mutant VirD2 gene (VirD2 Mutant 4 (T mutant or the truncated mutant)). The PDS2 gene was amplified by PCR and the amplicons were deep-sequenced by Wide-seq. The top row indicates the nucleotide number within the PDS2 amplicon and the numbers within the table are the percent mutations at each site surrounding the Cas9 cleavage site (between nucleotides 290 and 291 (bolded)). The percent mutations includes nucleotide substitutions, insertions, and deletions.

FIG. 18 is a table of sequence characteristics of the VirD2 mutants, including their ability to mediate transient and stable transformation relative to the wild-type VirD2 protein. *Because of a mutation in the stop codon, the VirD2 4E12 sequence is extended as indicated in SEQ ID NO: 22 and SEQ ID NO: 24. ** VirD2 Mutant 4 has a frameshift after nucleotide 979 that results in the inclusion of 18 additional amino acids (GSQRAQTKRHFRCFSPGD (SEQ ID NO: 29)) as compared to wild-type (see also SEQ ID NO: 25). The nucleotide sequence encoding the VirD2 Mutant 4 (SEQ ID NO: 26) correspondingly has an additional 125 nucleotides (SEQ ID NO: 30) after nucleotide 979 of the corresponding wild-type sequence.

FIG. 19 shows images of plates containing tumors on root segments from a stable transformation assay of wild-type (ecotype Col-0) Arabidopsis root segments infected with different concentrations (10⁷ cfu/ml or 10⁸ cfu/ml, as indicated) of Agrobacterium strains harboring wild-type or a Tr mutant VirD2 (VirD2 Mutant 4) Agrobacterium strain containing oncogenes on the T-DNA (top). The images were taken, and tumors scored, one (1) month post-inoculation. A table (bottom) is also shown that summarizes the resulting data from the two independent studies conducted as described in the top image of FIG. 19.

FIG. 21 is data summarized from a transient transformation assay of wild-type (ecotype Col-0) Arabidopsis root segments infected with different concentrations (10⁷ cfu/ml or 10⁸ cfu/ml, as indicated) of Agrobacterium strains harboring wild-type Agrobacterium tumefaciens strain EHA105 or A. tumefaciens EHA105 harboring a Tr mutant VirD2 incorporated via a Ti-plasmid. The A. tumefaciens EHA105 strains (both wild-type and VirD2 Tr mutant) also harbored the T-DNA binary vector pBISN1 containing a gusA-intron gene. The Agrobacterium was killed, and six (6) days post-inoculation, the root segments were stained with X-gluc to assess GUS activity.

FIG. 20 is a table of mutation frequency data surrounding the Cas9 cleavage site in the PDS2 gene of N. Benthamiana leaves four (4) days post-infiltration with an A. tumefaciens EHA105 strain containing either a wild-type (Wt) or a VirD2 Tr mutant gene on a Ti-plasmid. tobacco leaves were individually infiltrated by the two strains and, eight (8) days later, DNA was isolated and analyzed. The PDS2 gene was amplified by PCR and the amplicons were deep-sequenced by Wide-seq. The top row indicates the nucleotide relative to the Cas9 cleavage site and the numbers within the table are the percent mutations at each site surrounding the Cas9 cleavage site.

### DETAILED DESCRIPTION

For the purpose of promoting an understanding of the principles of the present disclosure, references will now be made to the embodiments illustrated in the drawings, and specific language will be used to describe the same. It will nevertheless be understood that no limitation of the scope of this disclosure is thereby intended.

Provided are mutant VirD2 Agrobacterium strains, expression constructs, and genome editing systems for gene editing in plant genomes. In particular, the mutant VirD2 Agrobacterium strains, expression constructs, and genome editing systems can be used, and particularly effective, in connection with transiently transforming a plant genome.

The present expression constructs, vectors, strains, and genome editing systems can also provide for a method for incorporating exogenous expressible nucleic acid in a plant. The recombinant expression constructs can be transformed using a mutant VirD2 Agrobacterium strain appropriate for a selected plant target (e.g., plant materials target).

### Agrobacterium Strains

The present disclosure provides a series of Agrobacterium strains comprising VirD2 mutants, including but not limited to, nucleotide substitutions, insertions, and deletions, that effect efficient transient, but at least 5-50 folds less stable transformation and/or no stable transformation of a plant.

Mutant VirD2 Agrobacterium strains are provided that allow for the transient transformation of plant material. The phrase “plant materials” encompasses all aspects of plants to be used for Agrobacterium-mediated transformation of plants including plant cells, leaves, roots, stems, buds, flowers, fruits, seeds, germinated seeds or plant tissues of any other parts, or whole plants.

In certain embodiments, the mutant VirD2 Agrobacterium strains have different efficiencies in transformation of plant materials as compared to corresponding strains with wild-type VirD2 genes. In certain embodiments, the Agrobacterium expressing a mutant VirD2 gene, and generate mutant VirD2 proteins, can efficiently lead transfer deoxyribonucleic acid (T-DNA) into a plant nucleus so that it can express its encoded genes, but not integrate into the plant itself.

According to well-known techniques, Agrobacterium strains can be used to transfer a single-strand form of T-DNA and Virulence (Vir) effector proteins into plant genomes (e.g., crop genomes). Agrobacterium mediated transfer of T-DNA material typically comprises: (1) in vitro recombination of genetic elements (at least one of which is of foreign origin) to produce an expression construct for selection of transformation, (2) insertion of this expression construct containing the foreign DNA into a T-DNA region of a binary vector, which can, for example, consist of several hundreds of base pairs of Agrobacterium DNA flanked by T-DNA border sequences, (3) transfer of the sequences located between the T-DNA borders (often accompanied by some or all of the additional binary vector sequences from Agrobacterium) into the plant cell, and (4) selection of stably transformed plant cells.

An “expression construct” is a vector, such as a recombinant vector, suitable for expression of a nucleotide sequence of interest in an organism (e.g., a plant). “Expression” refers to the production of a functional product. For example, expression of a nucleotide sequence can refer to transcription of the nucleotide sequence (e.g., translation of DNA into a precursor or mature protein). An expression construct can be a linear nucleic acid fragment, a plasmid (e.g., a circular plasmid), a viral vector, or, in some embodiments, an RNA (e.g., mRNA) capable of translation. An expression construct can comprise, for example, regulatory sequences of different origin and nucleotide sequences of interest, or regulatory sequences and nucleotide sequences of interest of the same origin but arranged in a manner different from that normally found in nature.

Accordingly, a plant can be created that expresses specifically desired traits, such as improved drought resistance or better nutritional content, which can be valued by growers and/or useful to industry. However, with conventional methodologies (such as the one outlined above), following transfer, the T-DNA is integrated into the plant chromosome and permanently express encoded transgenes, a process known as stable transformation, to result in a “transgenic” or genetically modified (GM) plant. “Stable transformation” refers to the introduction of an exogenous nucleotide sequence into the genome, resulting in stable inheritance of the exogenous nucleotide sequence. Once stably transformed, the exogenous nucleic acid sequence is stably integrated into the genome of the plant and any successive generation thereof. GM plants are highly regulated or even prohibited in certain jurisdictions, especially in connection with plants for use in the food supply.

The VirD2 mutant Agrobacterium strains, expression constructs, and genome editing systems hereof can modify plants to express one or more traits of interest using T-DNA and, while the plant genome may be altered, a transgenic plant is not created as the delivered T-DNA is either destroyed (e.g., by naturally existing enzymes in the plant cells) or diluted out of the plant nuclei as the cells divide. A “trait” means a physiological, morphological, biochemical, or physical characteristic of a cell or organism (e.g., plant). Accordingly, these mutant strains can leverage transient transformation processes to introduce traits of interest into the plant, without resulting in a transgenic plant.

“Transformation” of plant cells encompasses the placement of translationally functional nucleic acid(s) into a target plant cell via the expression constructions, mutant Agrobacterium strains, vectors, and/or methods hereof “Transient transformation” refers to the introduction of a nucleic acid molecule or protein into a cell to perform a function without stable inheritance of an exogenous nucleotide sequence. In transient transformation, the exogenous nucleic acid sequence is not integrated into the genome.

The VirD2 mutant Agrobacterium strains result in different efficiency in transformation of plants than that demonstrated by strains with wild-type VirD2 genes. In certain embodiments, the mutant VirD2 Agrobacterium strain shows about the same transient transformation as a strain harboring wild-type VirD2 genes.

Phenotype data such as transformation efficiency can be obtained by evaluating the expression of a marker gene and/or a selective marker gene co-introduced with a gene desired to be introduced into a plant. Marker genes and/or selective marker genes that can be used include, without limitation, the GUS (β-glucuronidase) gene and/or antibiotic resistance genes (e.g., PPT (phosphinothricin) resistance genes, hygromycin resistance genes, kanamycin resistance genes, paromoycin resistance genes, and the like). When the GUS gene is used as a marker gene, transformation efficiency can be evaluated from the coloration resulting from cleavage of X-gluc (5-bromo-4-chloro-3-indolyl-β-D-glucuronic acid) by GUS. When a gene resistant to an antibiotic is used as a selective marker gene, evaluation can be made from the extent of growth on a selective medium containing the antibiotic after transformation.

In certain embodiments, an Agrobacterium strain is provided that comprises a mutant VirD2 gene that effects transient transformation of a plant (e.g., efficient transient transformation of the plant or intermediate efficiency with respect to transient transformation). The phrase “efficient transient transformation” encompasses transient transformation as detectable by a standard assay which is about 75% to about 100% of that demonstrated by wild-type. The phrase “intermediate efficiency” as it relates to transient transformations encompasses transient transformation as detectable by a standard assay that is about 5% to about 50% of that demonstrated by wild-type.

The Agrobacterium strain can comprise any suitable Agrobacterium strain. The Agrobacterium strain can comprise an EHA105 derivative.

The mutant VirD2 strain can encode a wild-type VirD2 protein with certain modifications or mutations therein. “Mutation” or “mutagenesis” of a target region, for example, an amino acid residue or a VirD operon, means that the nucleic acid or amino acid sequence (as appropriate) as encoded by naturally occurring “wild-type” genes has been altered by mechanical, chemical, photonic, radiologic, enzymatic, or other means to change the nucleic acid sequence or amino acid sequence at one or more positions. With respect to a mutagenized nucleic acid sequence, it can encode a protein that is different in amino acid sequence from that which would result from translation of a wild-type gene or other nucleic acid sequence. “Mutagenized” or “mutation” can include a substitution, a chemical substitution of analog bases or residues, a stop-codon incorporation, an incorporation of additional bases or residues, a frame-shift mutation, and/or other such modifications.

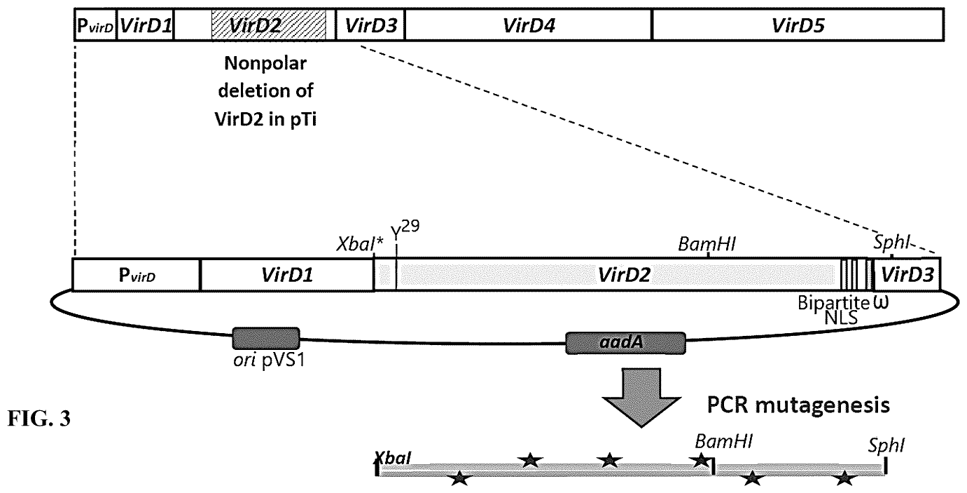

Non-limiting examples of a wild-type VirD2 protein can be or comprise SEQ ID NO: 16, SEQ ID NO: 17, or a functional variant of SEQ ID NO: 16 or SEQ ID NO: 17 (FIGS. 2A-2B). A wild-type VirD2 protein can be encoded by SEQ ID NO: 15, SEQ ID NO: 18, or a functional variant of SEQ ID NO: 15 or SEQ ID NO: 18 (FIGS. 2A-2B).

The modifications or mutations of the wild-type VirD2 can comprise a mutation at a threonine at position 319 of the wild-type VirD2 protein. The modifications or mutations of the wild-type VirD2 can comprise a combination of a mutation at a threonine at position 319 of the VirD2 protein, a mutation at a histidine at position 402 of the VirD2 protein, and an insertion of 30 to 40 amino acid residues at a terminal end the VirD2 protein. The modifications or mutations of the VirD2 can comprise an insertion of 1 to 20 amino acid residues after position 326 of the VirD2 protein, which affects a downstream frameshift.

When a position is specifically referenced in a protein, such position refers to the corresponding placement in the related wild-type protein to the extent it references a mutation thereof (e.g., a substitution, insertion, deletion, or the like).

In certain embodiments, the mutant Agrobacterium strain is the 4E12 strain comprising a mutation His⁴⁰²Arg, plus replacement of the stop codon with Gln extending the protein with the amino acids: Arg-Gln-Glu-Gly-Pro-Asn-Asn-Gly-Lys-Trp-Ser-Val-His-Asp-Thr-Leu-Cys-Trp-Pro-Gly-Leu-Arg-Arg-Thr-Asp-Gly-Arg-Thr-Pro-Trp-Ser-Arg-Ile-Arg-Leu (SEQ ID NO: 1).

In other embodiments, the mutant Agrobacterium strains are ExD3, containing Leu¹²²Gln and Ile⁹Val; 1B10 containing Glu⁶⁴Gly; 4G10 containing Lys³³⁸Asn and a frameshift after the stop codon expending the protein with sixteen (16) amino acids: Asp-Val-Met-Thr-Arg-Arg-Gln-Val-Arg-Ala-Glu-Gln-Thr-Val-Lys-Asp (SEQ ID NO: 2); 1G03, with Trp¹⁴¹stop codon; 21G2 with His¹⁵⁵Tyr and Lys¹⁶⁶Glu; 1 G03, with Trp¹⁴⁸Stop codon; 21 G2 with His¹⁵⁵Tyr and Lys¹⁶⁶Gu.

In certain embodiments, the mutant VirD2 strain (VirD2 ω Mutant) comprises mutations built into an omega (ω) mutant VirD2, which itself already has mutations (as compared to wild-type VirD2) comprising Asp⁴¹⁸Ser, Asp⁴¹⁹Ser, Gly⁴²⁰Ser, and Arg⁴²¹Ser. The additional mutations can include: 31V1-1/-2 with Lys³³²Met; 31C3 with Gly³⁶⁷Asp; 32C9 with Ser²⁷⁸Pro; 31C10 with Asn³⁷⁶Asp and Asn³⁴¹Ser; 31C11 with Lys³³²Glu; 31G7 with Ser³²⁰Pro; 36B1 with Asn⁴¹⁵Ser; 36B9-1 with Leu²⁷⁹Trp; 21F5 with Leu³⁸Arg; 311B7 with Val³³¹Ala; 31E7 with Asp³⁶¹Asn and Arg⁴¹³Ser; 32A2 with Thr³⁰Ala and Asp³⁸⁰Gly; 34A9 with Arg³³⁹Cys; 34E11 with Trp²¹⁹Ala and Arg¹⁸³Gln; 36A9 with Val³⁰⁵Ala; 36D10 with Pro³²²Leu; ωPCR-2 with Ser²⁸0Asn; ωPCR-76 with Ala³⁷⁹Val and Asp³⁸⁰His; ωPCR-77 with Ser³⁹⁴Pro; ωPCR-78 with Ile³⁰Ser; ωPCR-80 with Asn³⁶⁵Lys; ωPCR-84 with Thr³⁰²Ala; ωPCR-87 with Ile²⁹²Val; ωPCR-93 with Thr⁴²⁴Pro and missing C3⁶² causing a frameshift and extending the protein with the amino acids: Arg-Asp-Arg-Asp-Arg-Ile-Met-Ala-Asn-Gly-Gln-Phe-Thr-Ile-Arg-Ser-Ala-Gly-Pro-Ala-Ser-Val-Gly-Leu-Thr-Gly-Glu-Arg-Arg-Gly-Ala-Ala-Ser-Ala-Ser-Ser-Ser-Ala-Ser-Ser-Asn-Ala-Cys-Gln-Pro-Pro-Gln-Gly-Ser-Pro-Arg-Asp-Gln-Ser-Thr-Leu-Ile-Gln-Pro-Leu-Arg-Cys-Tyr-Ser-Ala-Val-Gly-Phe (SEQ ID NO: 3); ωPCR-127 with Arg²⁷⁶His and Arg⁴¹¹His; ωPCR-11 with Ala³⁵⁸Ser; ωPCR-20 with Asn³⁵¹Ser; ωPCR-16 with Lys³⁵³Glu; ωPCR-25 with Val²⁷⁷Ala; ωPCR-39 with Thr³⁸⁸Ser; ωPCR-45 with Ser³⁶²Pro; and ωPCR-55 with Lys³³⁸Gu.

In certain embodiments, the mutant Agrobacterium strains show transient GUS activity on infiltrated tobacco leaves and no or extremely low stable transformation of Kalanchoe and Arabidopsis.

The VirD2 protein can be or comprise SEQ ID NO: 19, SEQ ID NO: 23, SEQ ID NO: 25, or a functional variant of SEQ ID NO: 19, SEQ ID NO: 23, or SEQ ID NO: 25. The mutant VirD2 gene (e.g., that is expressed by the Agrobacterium strains hereof and encodes the VirD2 protein) can be or comprise SEQ ID NO: 20, SEQ ID NO: 24, SEQ ID NO: 26, or a functional variant of SEQ ID NO: 20, SEQ ID NO: 24, or SEQ ID NO: 26.

In certain embodiments, the mutant VirD2 gene of the Agrobacterium strain is preceded by an enhanced Shine-Dalgarno ribosome binding site sequence. Inclusion of a Shine-Dalgarno sequence in front of VirD2 can enhance translation of the mutagenized VirD2 proteins in Agrobacterium.

The Shine-Dalgarno sequence can be or comprise SEQ ID NO: 31 or a functional variant thereof. The term “functional variant” refers to a nucleotide, peptide, a polypeptide, or a protein having substantial or significant sequence identity or similarity to the reference nucleotide, peptide or polypeptide, which functional variant retains the biological activity of the reference sequence of which it is a variant. Functional variants encompass, for example, those variants of a sequence (the parent sequence) that retain the ability to exhibit the properties (such as, for example, binding functionality) and to a similar extent, the same extent, or to a higher extent, as the parent sequence. In reference to a nucleic acid sequence encoding the peptide or polypeptide, in some embodiments a nucleic acid sequence encoding a functional variant of the peptide or is about 10% identical, about 25% identical, about 30% identical, about 50% identical, about 65% identical, about 75% identical, about 80% identical, about 90% identical, about 95% identical, or about 99% identical to the nucleic acid sequence encoding the parent sequence.

In certain embodiments, the mutant VirD2 strain encodes a wild-type VirD2 protein except for: (i) a mutation at a threonine at position 319 of the VirD2 protein; (ii) a mutation at a threonine at position 319 of the VirD2 protein, a mutation at a histidine at position 402 of the VirD2 protein, an insertion of 30 to 40 amino acid residues at a terminal end the VirD2 protein due to a truncation mutation at a glutamine at position 425 (i.e., resulting in a mutation in the stop codon); or (iii) an insertion of 1 to 20 amino acid residues after position 326 of the VirD2 protein, which affects a downstream frameshift.

The mutant VirD2 strain can encode a wild-type VirD2 protein except for a mutation at a threonine at position 319 of the VirD2 protein. In certain embodiments, the mutation at position 319 of the wild-type VirD2 protein comprises a substitution of threonine with an amino acid other than threonine. In certain embodiments, the mutation at position 319 of the VirD2 protein is a substitution of threonine with alanine. In certain embodiments, the mutated VirD2 gene of the Agrobacterium strain comprises the nucleic acid sequence of mutant 4F03 (SEQ ID NO: 20 or a functional variant thereof) (VirD2 Mutant 1). In certain embodiments, the mutant VirD2 gene of VirD2 Mutant 1 encodes a protein that is or comprises SEQ ID NO: 19 or a functional variant thereof.

The mutant VirD2 strain can encode a wild-type VirD2 protein except for a mutation at a histidine at position 402 of the VirD2 protein. In certain embodiments, the mutation at position 402 of the VirD2 protein comprises a substitution of histidine with an amino acid other than histidine. In certain embodiments, the mutation at position 402 of the VirD2 protein is a substitution of histidine with arginine. This mutant VirD2 strain further comprises an insertion of 30 to 40 amino acid residues at a terminal end of the VirD2 protein resulting from a truncation mutation at a glutamine at position 425 (which results in a mutation of the stop codon). In certain embodiments, the insertion is the addition of an additional 38 amino acid residues at the terminal end of the VirD2 protein. The insertion can be or comprise SEQ ID NO: 32 or a functional variant thereof.

In certain embodiments, the mutated VirD2 gene of the Agrobacterium strain comprises the nucleic acid sequence of mutant 4E12 (SEQ ID NO: 22 or a functional variant thereof) (VirD2 Mutant 2). In certain embodiments, the mutant VirD2 gene of VirD2 Mutant 2 encodes a protein that is or comprises SEQ ID NO: 21 or a functional variant thereof.

The mutant VirD2 strain can encode a wild-type VirD2 protein except for a combination of: (1) a mutation at a threonine at position 319 of the wild-type VirD2 protein, (2) a mutation at a histidine at position 402 of the wild-type VirD2 protein, and (3) an insertion of 30 to 40 amino acid residues at a terminal end of the VirD2 protein due to a truncation mutation at a glutamine at position 425 (e.g., resulting in a mutated stop codon). In certain embodiments, the mutation at position 319 of the VirD2 protein comprises a substitution of threonine with an amino acid other than threonine. In certain embodiments, the mutation at position 319 of the VirD2 protein is a substitution of threonine with alanine. In certain embodiments, the mutation at a histidine at position 402 of the VirD2 protein comprises a substitution of histidine with any amino acid other than histidine. In certain embodiments, the mutation at position 402 of the VirD2 protein is a substitution of histidine with arginine. In certain embodiments, the insertion of 30 to 40 amino acid residues at a terminal end of the VirD2 protein results from a truncation mutation of a glutamine at position 425, which translates to a mutation of the related stop codon. Such a stop codon can be the final stop codon of the VirD2 gene. In certain embodiments, the insertion is the addition of an additional 31 to 39 amino acid residues at the terminal end of the VirD2 protein. In certain embodiments, the insertion is the addition of an additional 32 to 38 amino acid residues at the terminal end of the VirD2 protein. In certain embodiments, the insertion is the addition of an additional 33 to 37 amino acid residues at the terminal end of the VirD2 protein. In certain embodiments, the insertion is the addition of an additional 34 to 36 amino acid residues at the terminal end of the VirD2 protein. The ranges specified in this paragraph are inclusive of the end points stated and all 1 amino acid residue increments encompassed thereby.

In certain embodiments, the insertion is the addition of an additional 35 amino acid residues at the terminal end of the VirD2 protein. In certain embodiments, the insertion is the addition of an additional 38 amino acid residues at the terminal end of the VirD2 protein. The insertion of 30 to 40 amino acid residues at a terminal end can be or comprise SEQ ID NO: 32 or a functional variant thereof.

In certain embodiments, the mutated VirD2 gene of the Agrobacterium strain comprises the nucleic acid sequence of mutant 4E12+4F03 or pE4905 or “Double Mutant” (SEQ ID NO: 24 or a functional variant thereof) (VirD2 Mutant 3). In certain embodiments, the mutant VirD2 gene of VirD2 Mutant 3 encodes a protein that is or comprises SEQ ID NO: 23 or a functional variant thereof.

The mutant VirD2 strain can encode a wild-type VirD2 protein except for an insertion of 1 to 20 amino acid residues after position 326 of the VirD2 protein. This insertion can be immediately after position 326 of the VirD2 protein and can affect a downstream frameshift. In certain embodiments, the insertion is the addition of an additional 2 to 19 amino acid residues immediately after position 326 of the VirD2 protein. The insertion after position 326 of the VirD2 protein can be the addition of 3 to 18 amino acid residues immediately after position 326. The insertion after position 326 of the VirD2 protein can be the addition of 4 to 17, 5 to 16, 6 to 15, 7 to 14, 8 to 13, 9 to 12, 10, or 11 amino acid residues immediately after position 326. The insertion after position 326 of the VirD2 protein can be the addition of 18 amino acid residues immediately after position 326. The ranges specified in this paragraph are inclusive of the end points stated and all 1 amino acid residue increments encompassed thereby.

In certain embodiments, the insertion after position 326 of the VirD2 protein is or comprises SEQ ID NO: 30 or a functional variant thereof. The insertion of 1 to 20 amino acid residues after position 326 of the VirD2 protein can be encoded by SEQ ID NO: 29 or a functional variant thereof.

In certain embodiments, the mutated VirD2 gene of the Agrobacterium strain comprises the nucleic acid sequence of mutant pE4896_w34A2/truncated+extra or pE4960 or “truncated mutant” or “Tr mutant” (SEQ ID NO: 26 or a functional variant thereof) (VirD2 Mutant 4). In certain embodiments, the mutant VirD2 gene of VirD2 Mutant 4 encodes a protein that is or comprises SEQ ID NO: 25 or a functional variant thereof.

In certain embodiments, the VirD2 protein comprises a non-molar mutation.

Stable transformation of a target plant cell can be moderated when using the expression constructs and mutant VirD2 Agrobacterium strains. In certain embodiments, the mutant VirD2 Agrobacterium strains can have no stable transformation of a plant (e.g., a target plant cell). The phrases “no stable transformation” and “low stable transformation” mean a level of plant cell transformation that is not significantly detectable by routine assay. Thus the phrase “no stable transformation” is equivalent to from 0% to about 10% of wild-type. The phase “low stable transformation” is equivalent to from about 1% to about 25% of wild-type.

With regard to the desired effect of having lower to almost no detectable stable transformation of a target plant cell, in certain embodiments, the plant cells can be inoculated with a dose of the mutant VirD2 Agrobacterium strain(s) from about 10⁶ cfu/ml to about 10⁹ cfu/ml (such as 10⁶ cfu/ml, 10⁷ cfu/ml, 10⁸ cfu/ml, or 10⁹ cfu/ml). Inoculation with less than about 10⁷ cfu/ml can be effective for transient transformation with low or almost no stable transformation. In certain embodiments, administration of the mutant VirD2 Agrobacterium strains at concentrations approaching 10⁹ cfu/ml can achieve virtually no stable transformation in the target plant cell. Other transformation concentrations can yield intermediate levels of transformation as desired and demonstrated by the concentration dependent response of the expression constructs, vectors, and strains described.

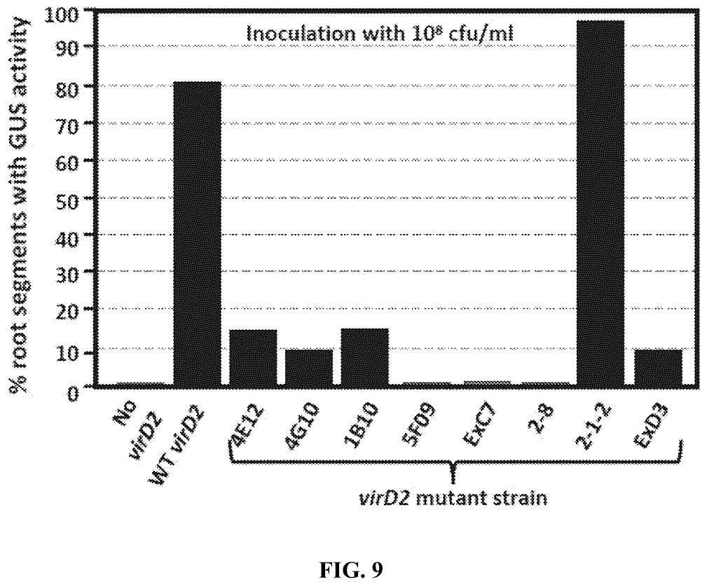

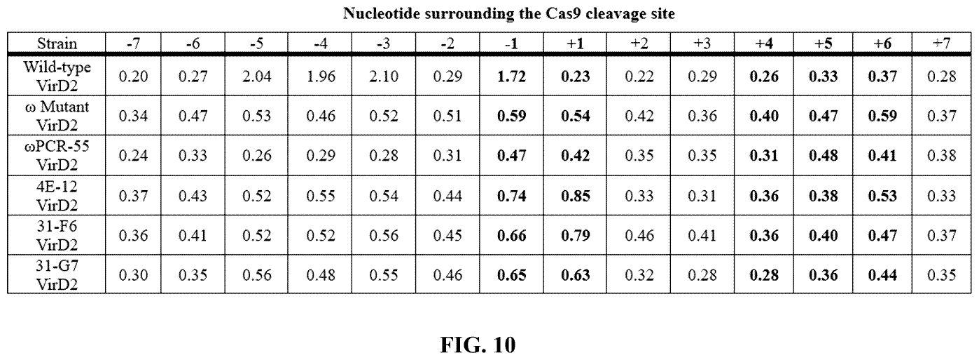

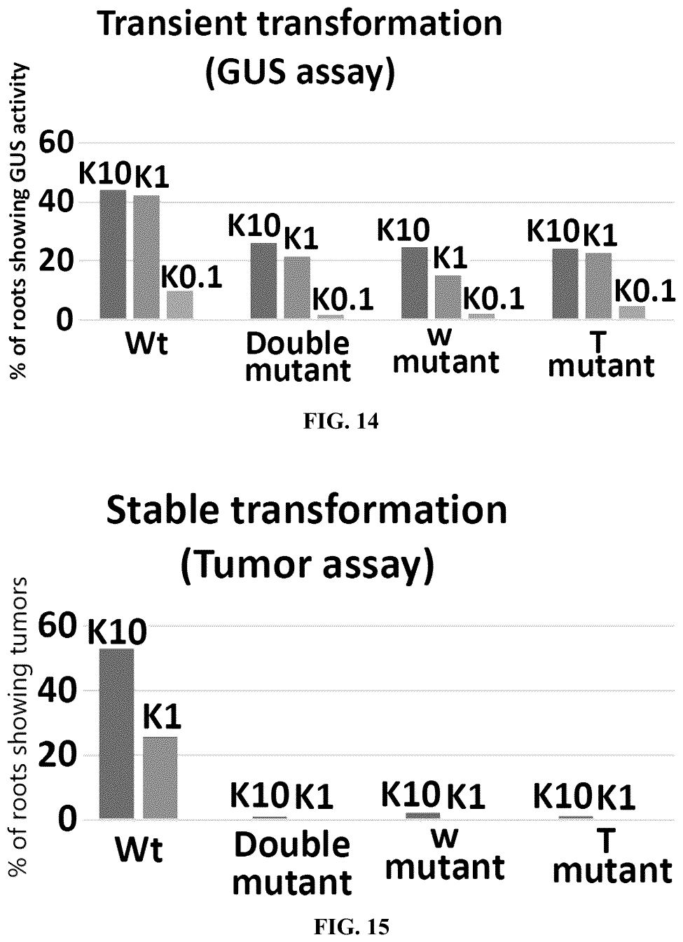

The mutant VirD2 Agrobacterium strains can have at least less stable transformation of a plant as compared to an Agrobacterium comprising a wild-type VirD2 genes (e.g., as measured by a tumor assay in FIGS. 12-13). In certain embodiments, a mutant VirD2 Agrobacterium strain that incorporates the gene VirD2 Mutant 1 can edit to about a 10% to about a 30% greater extent than does wild-type VirD2 (FIG. 16). In certain embodiments, a mutant VirD2 Agrobacterium strain that incorporates the gene VirD2 Mutant 2 can edit to about a 30% to about a 45% less extent as does wild-type VirD2 but has almost no stable transformation activity. In certain embodiments, a mutant VirD2 Agrobacterium strain that incorporates the gene VirD2 Mutant 3 (i.e., combining the mutations of VirD2 Mutants 1 and 2) can edit to about a 55% to about a 70% of that of wild-type VirD2, but has almost no stable transformation activity.

In certain embodiments, the mutant VirD2 Agrobacterium strains can transiently transform a host plant cell about 30% to about 70% as well as compared to an Agrobacterium wild-type strain (such as 30% to about 70%, about 30% to 70%, or 30%-70% as well). In certain embodiments, the mutant VirD2 Agrobacterium strain transiently transforms a host plant cell about 35% to about 65% as well as compared to an Agrobacterium wild-type strain (such as 35% to about 65%, about 35% to 65%, or 35%-65% as well). In certain embodiments, the mutant VirD2 Agrobacterium strain transiently transforms a host plant cell about 40% to about 60% as well as compared to an Agrobacterium wild-type strain (such as 40% to about 60%, about 40% to 60%, or 40%-60% as well). In certain embodiments, the mutant VirD2 Agrobacterium strain transiently transforms a host plant cell about 45% to about 55% as well as compared to an Agrobacterium wild-type strain (such as 45% to about 55%, about 45% to 55%, or 45%-55% as well). In certain embodiments, the mutant VirD2 Agrobacterium strain transiently transforms a host plant cell about 50% as well as compared to an Agrobacterium wild-type strain (such as 50% as well). All ranges set forth in this paragraph are inclusive of the stated end points and all 1% increments encompassed thereby.

The mutant VirD2 Agrobacterium strains can be incorporated into an expression construct (described above). In addition to the above description, an expression construct can be any suitable plasmid. A “suitable plasmid” encompasses the ordinary meaning of the term “plasmid,” and in general means a nucleic acid construct that is suitable for carrying operably inserted nucleic acids and functionally incorporating such nucleic acids into a targeted cell (e.g., a targeted plant cell). A suitable plasmid can contain nucleic acid sequences of any organism origin and can be amendable to molecular editing by enzymatic and physical tools known in the art.

In certain embodiments, the expression construct is a replicating plasmid. The expression construct can be a root-inducing plasmid (Ri-plasmid. The term “Ri-plasmid” includes all plasmids related to and that fall within the class of root-inducing plasmids seen in Agrobacterium. Ri-plasmids can induce hairy root diseases in dicots, and the virulence plasmid is named with pRi. The T-DNA of a Ri-plasmid can infect plant materials. A Ri-plasmid can integrate and express its bacterial genome within plant materials.

The expression construct can be a tumor-inducing plasmid (Ti-plasmid). The term “Ti-plasmid” includes all plasmids related to and that fall within the class of tumor-inducing plasmids seen in Agrobacterium. Ti-plasmids are typically named pTi- and can be used to integrate genes of interest into the genome of a specific plant material. The Ti-plasmid can be a pTiEHA105 plasmid.

In certain embodiments, a wild-type VirD2 gene of the expression construct (e.g., Ti-plasmid) is disarmed. In certain embodiments, the wild-type VirD2 gene of the expression construct is inactive, deleted, disruption, disarmed, and/or replaced by the mutant VirD2 gene. In certain embodiments of a mutant Agrobacterium strain, a mutant VirD2 gene is incorporated into a pTiEHA105 plasmid, wherein the wild-type VirD2 gene is inactive and non-functional. In certain embodiments of a mutant Agrobacterium strain, a mutant VirD2 gene is incorporated into a pTiEHA105 plasmid, wherein the wild-type VirD2 gene is inactivated, deleted, or replaced (e.g., by the mutant VirD2 gene). In certain embodiments of a mutant Agrobacterium strain, a mutant VirD2 gene is incorporated into a pTiEHA105 plasmid and replaces and/or disrupts the wild-type VirD2 gene. In certain embodiments of a mutant Agrobacterium strain, a mutant VirD2 gene is incorporated into a Ti- or Ri-plasmid and replaces and/or disrupts the wild-type VirD2 gene.

### Expression Constructs

Expression constructs are also provided that leverage the mutant VirD2 genes. In certain embodiments, the expression construct comprises a nucleic acid construct incorporating any of the mutant VirD2 genes described herein. The expression construct can comprise any of the constructs described. In certain embodiments, the expression construct comprises a recombinant DNA construct. In certain embodiments, the expression construct comprises an Ri-plasmid or a Ti-plasmid. In certain embodiments, the Ti-plasmid is or comprises a pTiEHA105 plasmid.

For example, an expression construct can comprise a mutant VirD2 gene that encodes a wild-type Agrobacterium VirD2 protein except for:
(i) a mutation at a threonine at position 319 of the VirD2 protein;
(ii) a mutation at a threonine at position 319 of the VirD2 protein, a mutation at a histidine at position 402 of the VirD2 protein, and an insertion of 30 to 40 amino acid residues at a terminal end of the VirD2 protein resulting from a truncation mutation at a glutamine at position 425; or
(iii) an insertion of 1 to 20 amino acid residues after position 326 of the VirD2 protein, which affects a downstream frameshift. The wild-type VirD2 protein can be or comprise SEQ ID NO: 16 or SEQ ID NO: 17, for example.

In certain embodiments, the mutant VirD2 gene of the expression construct is preceded by an enhanced Shine-Dalgarno sequence. The Shine-Dalgarno sequence can be or comprise SEQ ID NO: 31 or a functional variant of SEQ ID NO: 31.

The expression construct can comprise a nucleic acid sequence where the nucleic acid codon for amino acid threonine at position 319 (Thr³¹⁹) of the VirD2 protein encodes for an amino acid other than threonine. In certain embodiments, the nucleic acid sequence comprises a nucleic acid codon at position 319 of the VirD2 protein that encodes for alanine (i.e., the wild-type VirD2 comprises a substitution at Thr³¹⁹Ala). The expression construct can comprise a Ti- or Ri-plasmid having a mutant VirD2 gene that is or comprises SEQ ID NO: 19 (VirD2 Mutant 1) or a functional variant thereof.

The expression construct can comprise a nucleic acid sequence that encodes a wild-type Agrobacterium VirD2 protein except that the nucleic acid codon for amino acid histidine at position 402 of the VirD2 protein encodes for an amino acid other than histidine (i.e., the wild-type VirD2 comprises a substitution at His⁴⁰²Arg), and inserts 30 to 40 amino acid residues at a terminal end of the VirD2 protein (e.g., due to a truncation mutation at a glutamine at position 425 that correlates with—and modifies—a stop codon). The expression construct can comprise a Ti- or Ri-plasmid having a mutant VirD2 gene that is or comprises SEQ ID NO: 22 (VirD2 Mutant 2) or a functional variant thereof.

The expression construct can comprise a nucleic acid sequence where: (i) the nucleic acid codon for amino acid threonine at position 319 (Thr³¹⁹) of the VirD2 protein encodes for an amino acid other than threonine; (ii) the nucleic acid codon for amino acid histidine at position 402 of the VirD2 protein encodes for an amino acid other than histidine and there is an insertion of 30 to 40 amino acid residues at a terminal end of the VirD2 protein (e.g., at position 425 of the VirD2 protein); and (iii) an insertion of 30 to 40 amino acid residues at a terminal end of the VirD2 protein is encoded, which results from a truncation mutation of a glutamine at position 425.

In certain embodiments, the nucleic acid sequence comprises a nucleic acid codon at position 319 of the VirD2 protein that encodes for alanine (i.e., a substitution at Thr³¹⁹Ala), and a nucleic acid codon at position 402 that encodes for arginine (i.e., a substitution at His⁴⁰²Arg), and the additional amino acid residues (e.g., 38 amino acid residues) due to the truncation at amino acid 425 Gln of the VirD2 protein are or comprise SEQ ID NO: 30 or a functional variant thereof. The expression construct can comprise a Ti- or Ri-plasmid having a mutant VirD2 gene that is or comprises SEQ ID NO: 24 (VirD2 Mutant 3) or a functional variant thereof.

The expression construct can comprise a nucleic acid sequence that encodes a wild-type Agrobacterium VirD2 protein except that there is truncation and an insertion of 1 to 20 amino acid residues after position 326 of the VirD2 protein (e.g., comprising 18 amino acid residues inserted immediately after position 326), which results in a frameshift downstream of the insertion. In certain embodiments, the inserted amino acid residues are or comprise SEQ ID NO: 29 or a functional variant thereof. The expression construct can comprise a Ti- or Ri-plasmid having a mutant VirD2 gene that is or comprises SEQ ID NO: 26 (VirD2 Mutant 4) or a functional variant thereof, which includes SEQ ID NO: 30 after position 979 of the VirD2 gene and creates a frameshift downstream of such insertion.

The expression construct can comprise a recombinant DNA construct comprising one or more mutant VirD2 genes. In certain embodiments, the expression construct comprises a nucleic acid sequence of a Ti- or Ri-plasmid having a VirD2 gene that is or comprises VirD2 Mutant 1 and VirD2 Mutant 4. A mutant VirD2 gene can be incorporated into an expression construct (e.g., a recombinant DNA construct) in suitable configurations as to provide for the interruption of, disruption of, or otherwise rendering the wild-type VirD2 gene inoperative and/or inactive.

The expression construct can additionally comprise operating regulatory segments that can enhance or otherwise allow for regulation of gene expression or T-DNA formation. The operating regulatory segments can comprise regions that regulate production of the mutant VirD2 gene product, whether or not such regulatory sequences are adjacent to coding and/or transcribed sequences. In certain embodiments, the operating regulatory segments can be one or more of a promoter sequence, a terminator, a translational regulatory sequence such as ribosome binding sites and/or internal ribosome entry sites (e.g., an enhanced Shine-Dalgarno sequence), enhancers, silencers, insulators, boundary elements, replication origins, matrix attachment sites, and locus control regions.

Further, will be understood that the disclosure is presented in this manner merely for explanatory purposes and the principles and embodiments described herein may be applied to constructs that have configurations other than as specifically described herein. Indeed, it is expressly contemplated that the components of the constructs of the present disclosure may be tailored in furtherance of the desired application thereof.

### Genome Editing Systems

The Agrobacterium mutant strains, mutant genes, and expression constructs can be used for genome engineering. The phrase “used for genome engineering” refers to the incorporation of targeting nucleic acid sequences, nucleic acid markers, and other such gene editing components into the expression constructs and Agrobacterium strains hereof Such genomic editing tools include, without limitation, CRISPR (Clustered Regularly Interspaced Short Palindromic Repeats) nucleic acid sequences. The expression constructs, mutant Agrobacterium strains, and mutant genes can be useful in an analogous way as the CRISPR-Cas9 genome editing tool for editing plant genomes.

Also provided are genome editing systems that comprise the mutant Agrobacterium VirD2 gene(s) and/or expression constructs hereof. In certain embodiments, the genome editing system comprises any of the expression constructs described. In certain embodiments, the genome editing system comprises an expression construct comprising a mutant Agrobacterium VirD2 gene that encodes an Agrobacterium wild-type VirD2 protein except for: (i) a mutation at a threonine at position 319 of the VirD2 protein, (ii) a mutation at a threonine at position 319 of the VirD2 protein, a mutation at a histidine at position 402 of the VirD2 protein, and an insertion of 30 to 40 amino acid residues at a terminal end of the VirD2 protein resulting from truncation of a glutamine at position 425 of the VirD2 protein, or (iii) an insertion of 1 to 20 amino acid residues after position 326 of the VirD2 protein, which affects a downstream frameshift.

The genome editing system further comprises a sequence-specific nuclease, a DNA polymerase, and/or a DNA polymerase recruitment protein, or an expression construct comprising a nucleotide sequence encoding the sequence-specific nuclease, the DNA polymerase and/or the DNA polymerase recruitment protein.

Targeted CRISPR-Cas9 mechanisms are known in the art and can be incorporated into the genome editing system hereof. The sequence specific nuclease can be a CRISPR nuclease, such as a CRISPR nickase. The CRISPR nickase can be a Cas9 nickase. In certain embodiments, the expression construct comprising a nucleotide sequence encoding the sequence-specific nuclease comprises a T-DNA binary vector and the sequence specific nuclease is a Cas9 nickase.

The DNA polymerase can comprise (or be part of) a primer-based PCR system as is known in the art.

The genome editing system can further comprise a guide RNA and/or an expression construct comprising a nucleotide sequence encoding the guide RNA. In certain embodiments, the guide RNA targets a PDS2 gene of Nicotiana benthamiana. In certain embodiments, the genome editing system further comprises a guide RNA protospacer that targets a PDS2 gene of N. benthamiana, wherein the guide RNA protospacer has or comprises SEQ ID NO: 12.

In certain embodiments of the genome editing system the mutant VirD2 gene of the expression construct is or comprises SEQ ID NO: 26 or a functional variant of SEQ ID NO: 26, and the gene editing system, in use, affects CRISPR mutagenesis about 50% to about 80% as well as compared to a genome editing system employing a wild-type Agrobacterium VirD2 strain (such as about 50% to 80%, 50% to about 80%, or 50% to 80% as well). In certain embodiments, the genome editing system affects CRISPR mutagenesis about 55% to about 75% as well as compared to a genome editing system employing a wild-type Agrobacterium VirD2 strain (such as about 55% to 75%, 55% to about 75%, or 55% to 75% as well). In certain embodiments, the genome editing system affects CRISPR mutagenesis about 60% to about 70% as well as compared to a genome editing system employing a wild-type Agrobacterium VirD2 strain (such as about 60% to 70%, 60% to about 70%, or 60% to 70% as well). In certain embodiments, the genome editing system affects CRISPR mutagenesis about 55% to about 78% as well as compared to a genome editing system employing a wild-type Agrobacterium VirD2 strain (such as about 55% to 78%, 55% to about 78%, or 55% to 78% as well). The ranges set forth in this paragraph are inclusive of the stated end points and include all 1% increments encompassed therein.

### Methods and Uses

A method of making and using the mutant Agrobacterium strains of the present disclosure are also provided herein. The present disclosure provides that the mutant Agrobacterium strains can be used for gene editing in any plant species. In certain embodiments, the mutant Agrobacterium strains are used CRISPR/Cas genome editing, and the VirD2 mutant of the mutant Agrobacterium strains support around the Cas9 cleavage site at at least 25-40% the frequency of the wide-type VirD2 gene. The present disclosure further provides that the mutant Agrobacterium strains can be used for altering, in a directed way, any particular nucleotide sequence through “homology-dependent repair (HDR).”

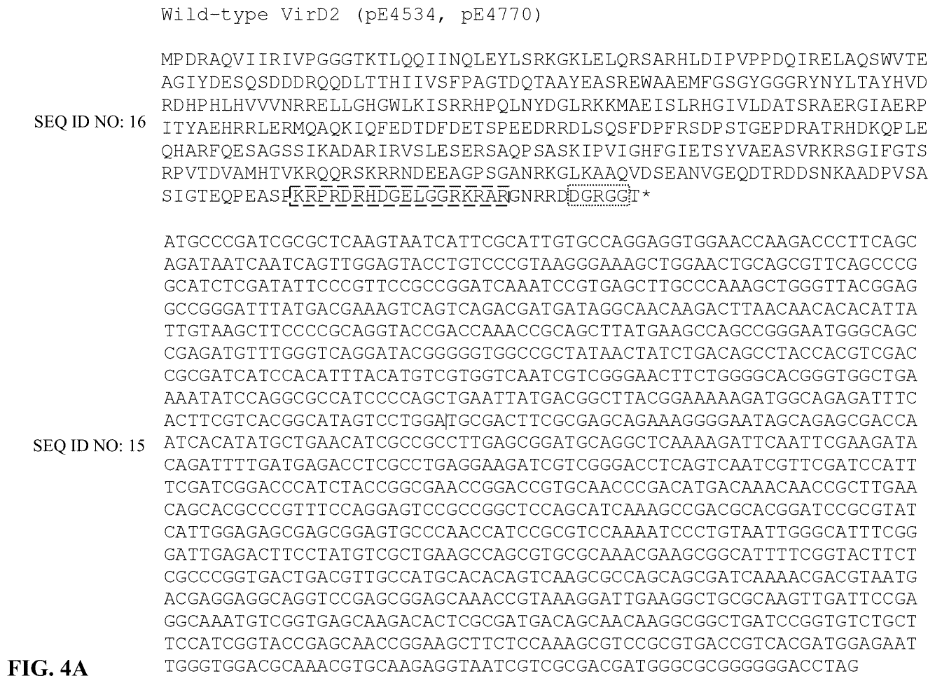

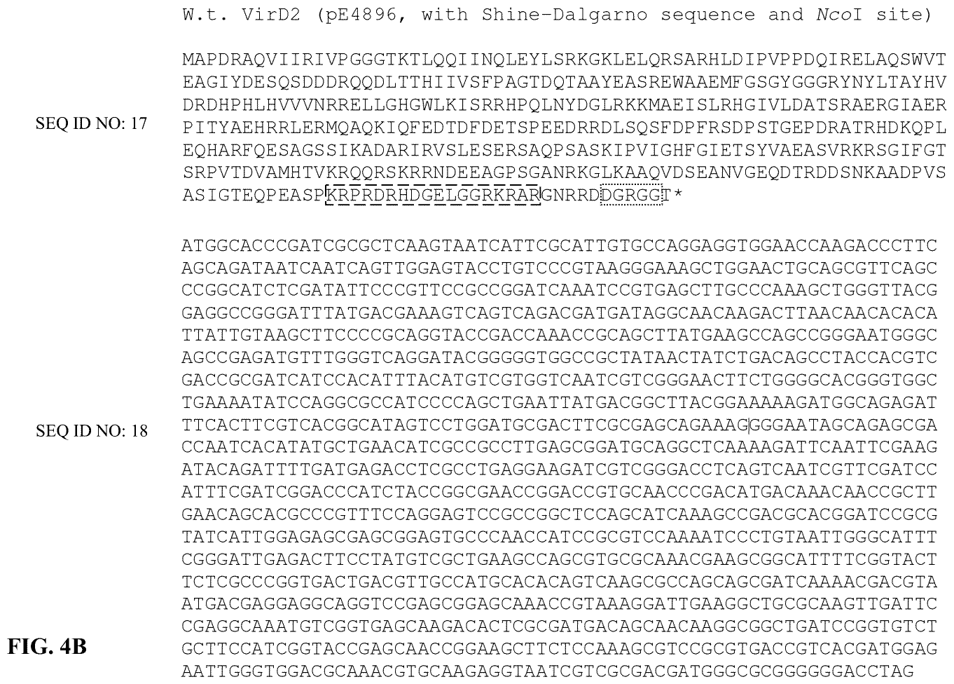

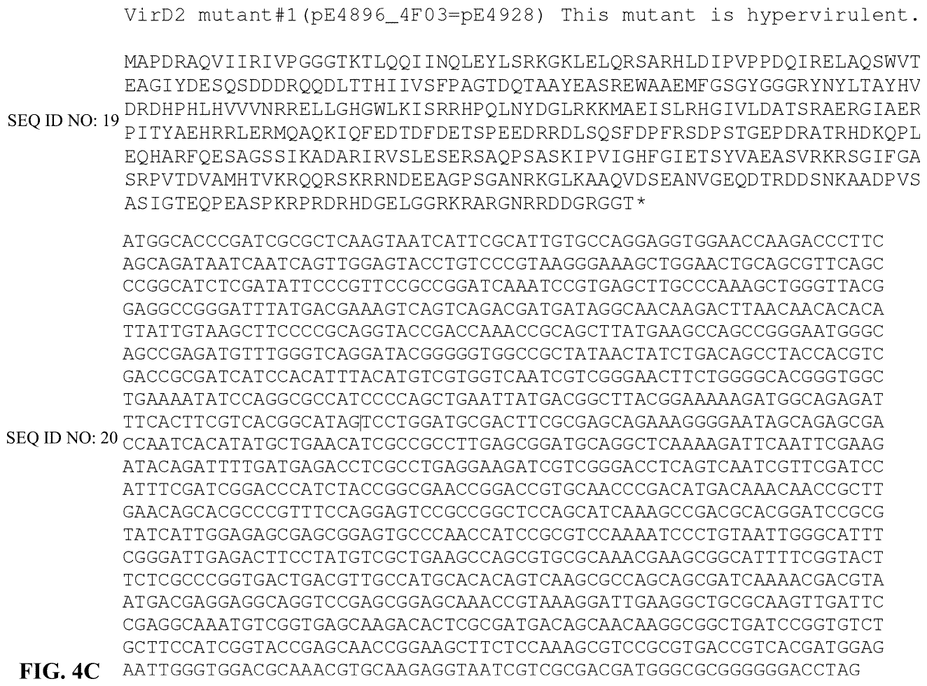

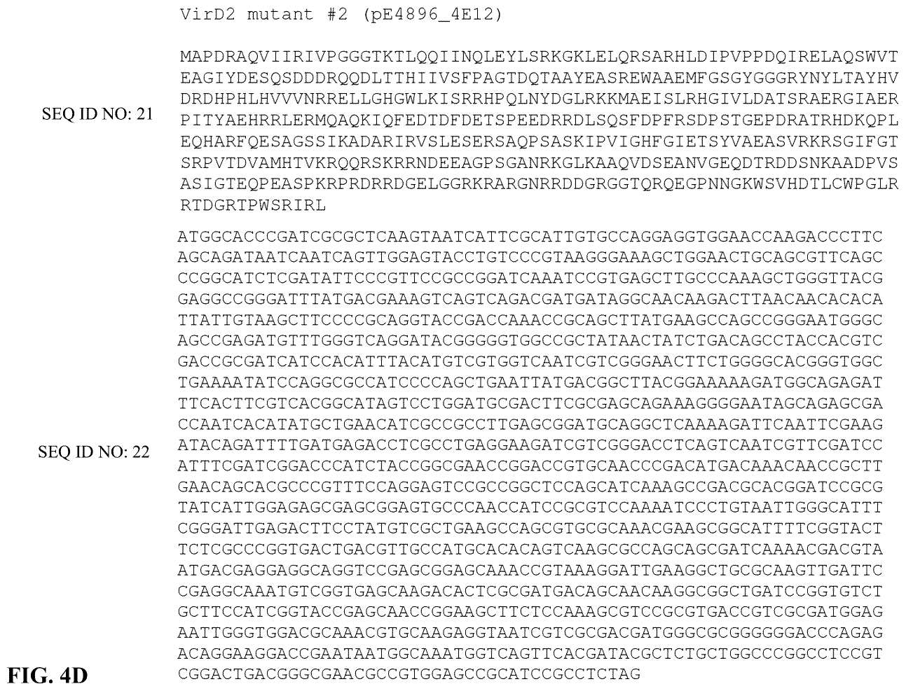

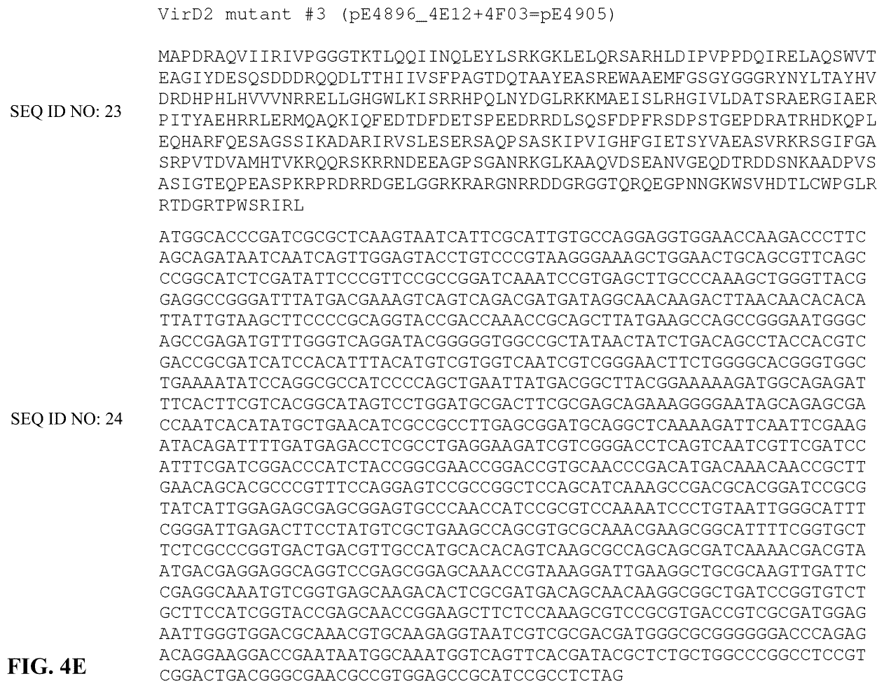

The present disclosure provides that by mutating a VirD2 gene and VirD2 protein, one can generate an Agrobacterium strain that can transfer but not integrate T-DNA. The process of generating such Agrobacterium strains is illustrated in FIG. 3 and the detailed descriptions of each step are provided as follows:
Step 1: Make an Agrobacterium strain containing a deletion of VirD2 that is non-polar (i.e., does not affect expression) on the downstream genes VirD3, VirD4, and VirD5. In this particular embodiment, a non-polar VirD2 mutation in the tumorigenic Ti-plasmid pTiA6 was obtained from Dr. Walt Ream; construction of this strain is described in Shurvinton et al., A nuclear localization signal and the C-terminal omega sequence in the Agrobacterium tumefaciens VirD2 endonuclease are important for tumor formation, Procedures Nat'l Academy Science 89: 11837-11841 (1992), that also indicates that nuclear localization signal and the C-terminal omega sequence in the Agrobacterium tumefaciens VirD2 endonuclease are important for tumor formation. A non-polar VirD2 mutation in the Ti-plasmid pTiEHA105 (in the Agrobacterium strain EHA105) was generated accordingly in the following way:
1. a 7.2 kbp XhoI fragment containing the entire agropine/succinamopine-type pTiBo542 virD operon from pE702 (the cosmid pEHC13) was subcloned into the XhoI site of pBluescript ks+ to make pE3332;
2. a 3.27 kbp Klenow-blunted SphI-XhoI fragment from pE3332 was subcloned into the SmaI-XhoI site of pE3351 (an Asp718 site filled pBluescript ks+) to make pE3353;
3. a 914 bp HindIII fragment from pE3052 (internal fragment of an octopine-type virD2 gene from pTiA6) was subcloned into a HindIII fragment deletion of pE3353 to make pE3355;
4. a 885 bp KpnI fragment (internal to the HindIII sites) from pE3355 was deleted to make pE3356;
5. an XhoI-NotI fragment (containing PvirD-virD1 partial VirD2 and VirD4) was also removed from pE3356 into pJQ200sk (a suicide plasmid, pE1416) to make pE3358; and
6. the above virD2 deletion operon (on pE3358) was recombined with the disarmed pTiBo542 (pTiEHA105) in Agrobacterium (using sacB counter-selection).
Step 2: Clone the VirDₚᵣₒₘₒₜₑᵣVirD1-VirD2 genes onto a plasmid that can replicate in both E. col and Agrobacterium. This was done by cloning an EcoRI-SphI fragment containing the VirDₚᵣₒₘₒₜₑᵣVirD1-VirD2 genes into pE4533, a plasmid containing a pVS1 origin of replication and the spectinomycin resistance gene AadA on the plasmid backbone. For some experiments, an XbaI site was added between sequences encoding the VirD1 stop codon and the VirD2 start codon. This was accomplished using PCR and the primers 5′-GACCATGATTACGAATCGAGC-3′ (SEQ ID NO: 4) and 5′-AATTTCTAGAGGGCACCTTTCAATAGCGAGC-3′ (SEQ ID NO: 5) to amplify the ProD-virD1 region of pWR160, adding an XbaI site in the VirD1-VirD2 intergenic region. The primer 5′-AATTTCTAGACTTGACCACGCACCTGACG-3′ (SEQ ID NO: 6) was then combined with the primer 5′-ATACGCGGATCCGTGCGTCGGC-3′ (SEQ ID NO: 7) to add, using PCR, the region of VirD2 upstream of the BamHI site to the ProD-virD1 region upstream, including then newly added XbaI site.
Step 3: Conduct PCR-based random mutagenesis of the virD2 gene from Step 2. This was done by using an expired OneTaq DNA polymerase (OneTaq® DNA Polymerase-New England Biolabs®). Primer 195 (5′-AATTTCTAGACTTGACCACGCACCTGACG-3′ (SEQ ID NO: 6)) was used in combination with Primer 196 (5′-ATACGCGGATCCGTGCGTCGGC-3′ (SEQ ID NO: 7)) to mutagenize the region of VirD2 upstream of the BamHI site. Primer 185 (5′-TTAAGTTGGGTAACGCCAGGG-3′ (SEQ ID NO: 8)) and Primer 186 (5′-AACAACCGCTTGAACAGCAC-3′ (SEQ ID NO: 9)) were used to mutagenize the C-terminal region of VirD2, downstream of the BamHI site.
Step 4: Sequence the various mutants to determine where mutations have occurred (FIG. 4). Sanger sequencing was conducted at the Purdue University Genomics Center and the Noble Foundation using the sequencing primer 5′-AATTTCTAGACTTGACCACGCACCTGACG-3′ (SEQ ID NO: 6), which flanks the 5′ end of VirD2, and 5′-ATTGCATGCATTGGACAGTGCAGAGCTAG-3′ (SEQ ID NO: 10), which (in reverse orientation) flanks the 3′ end of VirD2. Another primer, 5′-GAAGTCTCAATCCCGAAATGC-3′ (SEQ ID NO: 11), was used to sequence the mid-section of VirD2 upstream of the BamHI site.
Step 5: After missense mutations in virD2, that change the amino acid sequence, were identified, plasmids containing these mutant virD2 genes were individually introduced by electroporation into the non-polar virD2 mutant Agrobacterium strains (these non-polar virD2 mutations were built into a tumorigenic octopine-type strain, and into the disarmed strain A. tumefaciens EHA105). Each of the plasmids containing a mutant virD2 gene was individually introduced into these strains, along with appropriate T-DNA binary vectors to monitor transient and stable transformation (FIG. 5).
Step 6: The various Agrobacterium strains were used to inoculate wounded leaves of Kalanchoe diagremontiana. After one month, the wounds were scored for the formation of crown gall tumors (FIG. 6). This was accomplished as follows: Leaves of Kalanchoe diagremontiana approximately 10 cm long were surface sterilized with 70% ethanol. After the ethanol dried, sterile toothpicks were used to score the top epidermis of the leaf, not extending the wound through the entire leaf. As separate sterile toothpick was used to scrape each Agrobacterium strain from the agar surface of a petri dish containing YEP medium, and the resulting bacterial paste was applied to the wound (one strain per each wound). After 24 hr under low light in the laboratory, the plants were returned to a plant growth chamber to await tumor development.
Step 7. virD2 mutant Agrobacterium strains that did not incite tumors were tested for their ability to carry out transient transformation using a tobacco leaf infiltration assay. Because these bacteria contain the T-DNA binary vector pBISN1, successful transient transformation would generate GUS activity (as assayed by blue X-gluc staining). The results from some strains are shown in FIG. 7. Tobacco leaf infiltration was conducted as follows:
a) Separately grow the various Agrobacterium strains overnight at 30° C. with shaking in 50 ml YEP-medium plus the requisite antibiotics to maintain plasmids (kanamycin 50 mg/L and/or spectinomycin 100 mg/L);
b) measure A600 of the overnight culture on the next day;
c) Take out 2 ml cells, centrifuge, and resuspend the pellet into 2 ml of agroinfiltration buffer (10 mM MgCl₂, 10 mM MES buffer, pH 5.5-5.6) plus 200 mM acetosyringone to make the Aeoo=0.4-0.8. Keep the cells at room temperature for 2 hours;
d) Use a 1 ml syringe to infiltrate the underside of tobacco leaves;
e) On the following day, and on subsequent days, cut the infiltrated leaf region and place the tissue into a 1.5 ml microfuge tube. Add 0.5 ml of X-Gluc staining solution (50 mM NaPO₄, 10 mM EDTA, 0.1% Triton X-100, 1 mM X-gluc, pH 7.0) into the microcentrifuge tube; and
f) Incubate overnight at 37° C.
Step 8: After identifying Agrobacterium strains that did not elicit tumors on Kalanchoe but still effected good transient transformation of tobacco leaves, additional, more quantitative transformation assays were conducted on Arabidopsis roots. GUS activity (% of root segments staining blue with X-gluc) was used to investigate transient transformation, and generation of kanamycin-resistant calli or tumor formation to investigate stable transformation. Transient and stable Arabidopsis root segment transformation was carried out as described in Tenea et al., Overexpression of several Arabidopsis histone genes increases Agrobacterium-mediated transformation and transgene expression in plants, Plant Cell 21(10): 3350-3367 (2009). Overexpression of several Arabidopsis histone genes increases Agrobacterium-mediated transformation and transgene expression in plants.

Briefly, wild-type (ecotype Col-0) Arabidopsis seeds were surface sterilized, washed five times in sterile water, and placed at 4° C. overnight. The seeds were then plated onto solidified B5 medium plus 100 mg/L Timentin and germinated for 10-14 days until the seedlings had true leaves. Seedlings were then transferred to baby food jars containing solidified B5 medium and grown for a further 10-14 days. Roots of these plants were cut into 2-5 mM segments and placed, in a pile, onto solidified MS medium. 50 μl of the various concentrations (105 cfu/ml-10⁸ cfu/ml) of the different Agrobacterium strains were placed on the root segments, then the excess bacteria removed after 15 minutes. The plates were taped with plastic wrap and placed in a growth chamber (22° C.) for two days. For transient transformation, the root segments were moved as a pile to solidified CIM medium for 4 days, then stained for GUS activity as described above. For stable transformation, root segments were individually separated onto solidified MS medium plus 100 mg/L Timentin (for tumorigenesis assays) or solidified CIM medium plus 100 mg/L Timentin plus 100 mg/L kanamycin (for antibiotic resistance assays), the plates were taped with plastic wrap, and the plates incubated in a growth chamber (22° C.) for 4 weeks. Tumors, or kanamycin-resistant calli, were quantified using a low-power dissecting microscope.

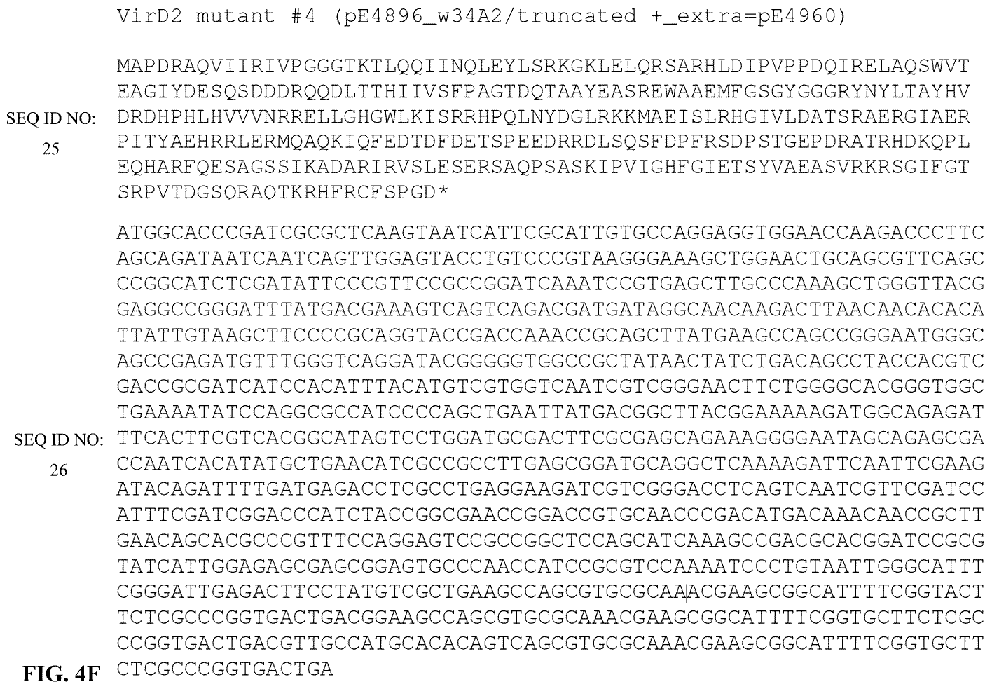

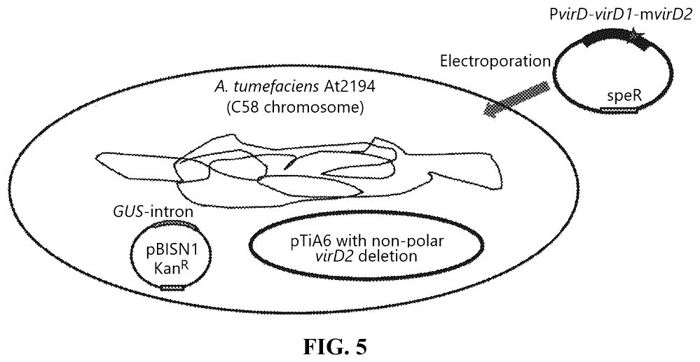

FIG. 8 shows an example of results from a stable transformation assay. Only one (2-1-2) of the tested virD2 mutant strains was efficient at stable transformation. FIG. 9 shows an example of results from a transient transformation assay. Note that mutant 5F09 did not show transient (or stable; FIG. 8) transformation activity, so it likely just contains a “dead” VirD2 protein. Mutant 4E12 has substantial transient transformation activity, making it a candidate for a strain that has very low stable but substantial transient transformation activity.
Step 9: Test the various Agrobacterium strains, containing mutant virD2 genes and a T-DNA binary vector containing within the T-DNA region a Cas9 gene and a gene encoding guide RNAs, for their ability to effect genome editing by causing mutations near the Cas9 cleavage site in plant genomes. This was done by cloning the protospacer 5′-GCTGCATGGAAAGATGATGA-3′ (SEQ ID NO: 12) (which targets the Nictotina benthamiana PDS1 and PDS2 genes) into the T-DNA region of the Agrobacterium strain At2331. At2331 is A. tumefaciens EHA105 with the non-polar virD2 mutation described above. It also contains the T-DNA binary vector pE4747, containing genes encoding Cas9, a Venus-NLS protein, a hptII hygromycin-resistance gene, and a sgRNA scaffold to express sgRNAs.

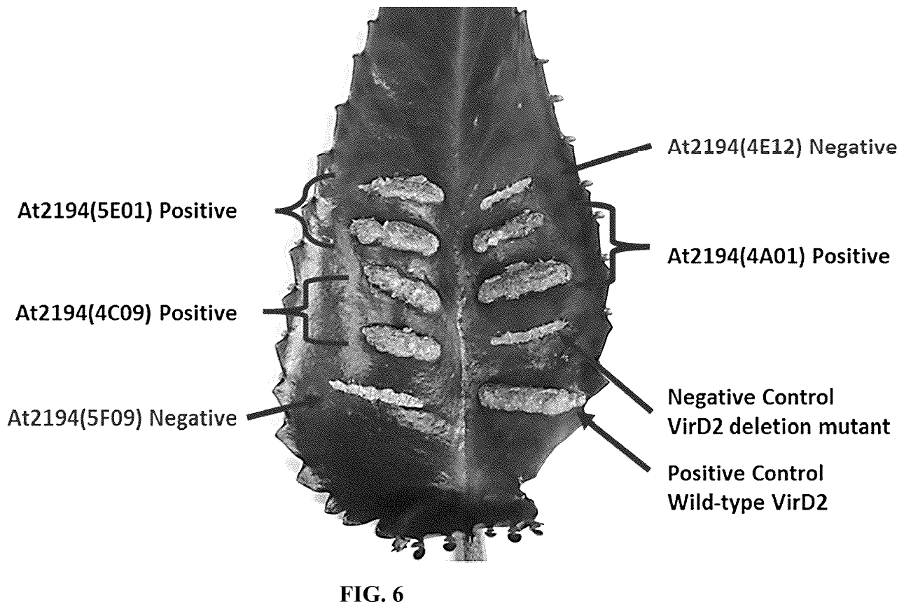

FIG. 10 shows examples of mutations caused by Cas9 cleavage in the N. benthamiana PDS2 gene and subsequent mis-repair of the double-strand DNA break.

It should be emphasized that the above descriptions of embodiments and/or special procedures/methods of the present disclosure are merely possible examples of implementations set forth for a clear understanding of the principles of the disclosure. Many variations and modifications may be made to the above-described embodiment(s) and/or procedures/methods without departing substantially from the spirit and principles of the disclosure. All such modifications and variations are intended to be included herein within the scope of this disclosure and protected by the following claims.

Also provided are methods for Agrobacterium-mediated incorporation of exogenous expressible nucleic acids into a host plant material. In certain embodiments of such methods, the method comprises infecting a target host plant material with an Agrobacterium strain described herein. In certain embodiments, infecting comprises inoculating the target host plant material with the Agrobacterium strain at a dose of at or between about 10⁶ cfu/ml to about 10⁹ cfu/ml.

Utilization of the methods hereof can be accomplished at various concentrations of Agrobacterium/expression construct depending upon selected conditions. Effective crop transformation experiments at a wide variety of concentrations, for example, up to 10⁹ cfu/ml, have been used in various transformation protocols.

As described above, altering the concentration of the infective vector (e.g., the mutant Agrobacterium strain or expression construct comprising the same) can change the resulting transient transformation efficiency. Higher concentrations of vector (see, e.g., FIGS. 12 and 13) generate different results as compared with lower levels. In certain embodiments, the methods comprise inoculating the target host plant material with the Agrobacterium strain at a dose of at or about 10⁶ cfu/ml. In certain embodiments, the methods comprise inoculating the target host plant material with the Agrobacterium strain at a dose of at or between about 10⁶ cfu/ml to about 10⁷ cfu/ml. In certain embodiments, the methods comprise inoculating the target host plant material with the Agrobacterium strain at a dose of at or about 10⁸ cfu/ml.

In certain embodiments, higher transformation can be achieved by inoculation with from about 10⁷ cfu/ml to about 10⁸ cfu/ml. The correlated effects between inoculation concentrations and efficacy, and the relationship between these factors, can be manipulated by altering the conditions and components utilized in the inoculation environment to achieve a desired effect.

In certain embodiments of the method, the Agrobacterium strain comprises a mutant VirD2 gene that is or comprises SEQ ID NO: 22 or a functional variant of SEQ ID NO: 22, and the dose is at or between about 10⁷ cfu/ml to about 10⁸ cfu/ml. In certain embodiments of the method, the Agrobacterium strain comprises a mutant VirD2 gene that is or comprises SEQ ID NO: 20, SEQ ID NO: 24, SEQ ID NO: 26, or a functional variant of SEQ ID NO: 20, SEQ ID NO: 24, or SEQ ID NO: 26, and the dose is at or between about 10⁷ cfu/ml to about 10⁸ cfu/ml.

In the methods, the Agrobacterium strain can comprise a mutant VirD2 gene that is or comprises SEQ ID NO: 26 or a functional variant of SEQ ID NO: 26, wherein the method can achieve at least 80- to 100-fold less stable transformation as compared to an Agrobacterium comprising a wild-type VirD2 gene or no stable transformation. In certain embodiments of the method where the Agrobacterium strain comprises a mutant VirD2 gene that is or comprises SEQ ID NO: 26 or a functional variant of SEQ ID NO: 26, and the host plant material can be transiently transformed about 30-70% as well as compared to when using an Agrobacterium wild-type strain.

Methods of transforming a target plant material are also provided, such methods comprising infecting a target plant cell, or plant, with an Agrobacterium strain hereof.

Transformed plant cells and/or plants which have been infected according to the methods hereof are also provided. Additionally, a plant comprising a plant material which has been infected according to a method hereof is also provided.

In addition, a method for genomic editing of the nucleic acid of a plant cell is provided, wherein such method comprises infecting a target plant cell with an Agrobacterium strain hereof, wherein said Agrobacterium strain comprises a gene-editing targeting system. Such gene-editing targeting systems may be primer-based PCR or targeted CRISPR-Cas9 mechanisms known in the art. Also provided is a plant which has been infected according to the method for effecting genomic-editing of the plant nucleic acid genes.

Still further, a use of the Agrobacterium strains hereof, the expression constructs hereof, and/or the genome editing systems hereof are provided, such uses to for transiently transforming a host plant cell of a plant such that the plant expresses one or more traits of interest.

### General

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

In the above description, numerous specific details are set forth to provide a thorough understanding of the present disclosure. Particular examples may be implemented without some or all of these specific details and it is to be understood that, unless otherwise stated, aspects hereof are not limited to particular biological systems or particular species of bacteria or plants, which can, of course, vary but remain applicable in view of the data provided herein.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

While aspects of the present disclosure can be described and claimed in a particular statutory class, such as the system statutory class, this is for convenience only and one of skill in the art will understand that each aspect of the present disclosure can be described and claimed in any statutory class.

It should be emphasized that the following disclosures are put forth so as to provide those of ordinary skill in the art with a complete disclosure and description of how the composition of matter, e.g., the mutant strains in this disclosure, and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein.

As used in the specification and the appended claims, the singular forms “a,” “an” and “the” include plural referents unless the context clearly dictates otherwise. Thus, for example, reference to “a short chain fatty acid,” “a carnitine derivative,” or “an adjuvant,” includes, but is not limited to, combinations of two or more such short chain fatty acids, carnitine derivatives, or adjuvants, and the like.

The terms “about,” “approximate,” “at or about,” and “substantially,” when referring to a number or a numerical value or range (including, for example, whole numbers, fractions, and percentages), means that the number or numerical range referred to is an approximation within experimental variability (or within statistical experimental error). That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±1%-15% variation of the stated number or numerical range (e.g., +/−5% to 15% of the recited value), provided that one of ordinary skill in the art would consider equivalent to the recited value (e.g., having the same function or result) and unless otherwise indicated or inferred. Similarly, when values are expressed as approximations, by use of the antecedent “about,” it will be understood that the particular value forms a further aspect. For example, if the value “about 10” is disclosed, then “10” is also disclosed.

When ratios, ranges, concentrations, amounts, and other numerical data are expressed herein in a range format, all combinations and sub-combinations of such ranges and specific embodiments therein are intended to be included. It will be further understood that the endpoints of each of the ranges are significant both in relation to the other endpoint, and independently of the other endpoint.

Additionally, it is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

The disclosure may be suitably practiced in the absence of any element(s) or limitation(s), which is/are not specifically disclosed herein. Thus, for example, each instance herein of any of the terms “comprising,” “consisting essentially of,” and “consisting of” (and related terms such as “comprise” or “comprises” or “having” or “including”) can be replaced with the other mentioned terms. Likewise, the singular forms “a,” “an,” and “the” include plural references unless the context clearly dictates otherwise. Thus, for example, references to “the method” include one or more methods and/or steps of the type, which are described and/or which will become apparent to those ordinarily skilled in the art upon reading the disclosure. The term “substantially” can allow for a degree of variability in a value or range, for example, within 90%, within 95%, or within 99% of a stated value or of a stated limit of a range.

It is recognized that various modifications are possible within the scope of the disclosure. Thus, although the present disclosure has been specifically disclosed in the context of preferred embodiments and optional features, those skilled in the art may resort to modifications and variations of the concepts disclosed herein. Such modifications and variations are considered to be within the scope of the disclosure as claimed herein.

It is therefore intended that this description and the appended claims will encompass all modifications and changes apparent to those of ordinary skill in the art based on this disclosure. Additionally, in describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

Further, the use of headings and subheadings is for ease of reference, given the length of the document. Description under one heading or subheading (such as a subheading in the Detailed Description) is not intended to be limited to only the subject matter set forth under that particular heading or subheading.

### ADDITIONAL EXAMPLES

The following examples serve to illustrate aspects of the present disclosure. The examples are not intended to limit the scope of the claimed invention in any way.

### Example 1

### Generation of Wild-Type VirD2-Deleted Agrobacterium Strain

VirD2 genes were targeted for mutagenesis, with the goal of generating mutant VirD2 proteins that efficiently lead T-DNA into a plant nucleus so that the plant can express the encoded genes, but not integrate.

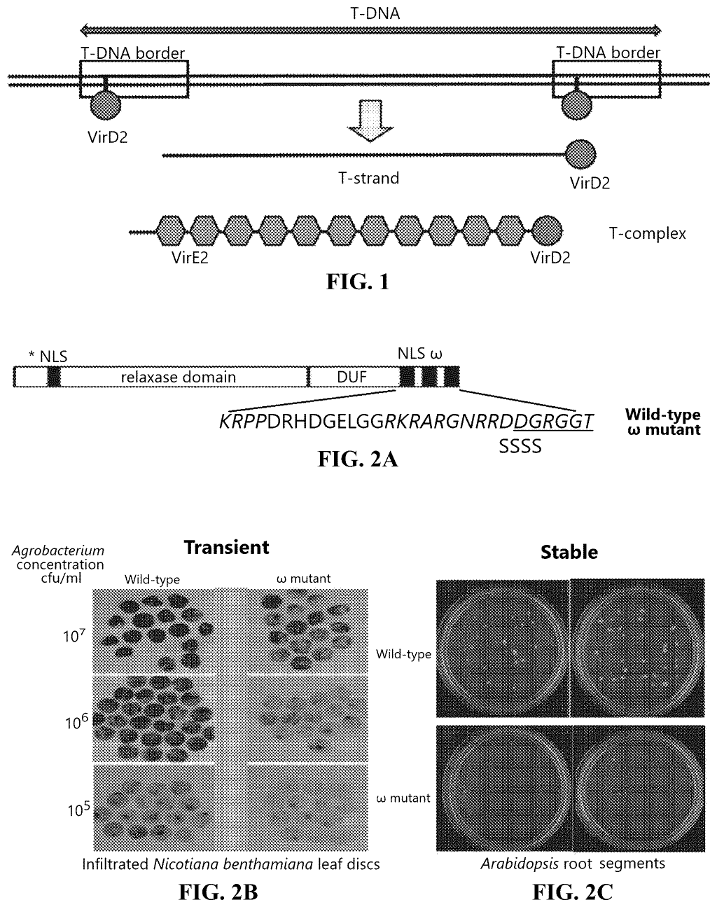

A mutant Agrobacterium strain was generated, in a A. tumefaciens EHA105 background, that contained a non-polar (on VirD3, VirD4, and VirD5) VirD2 deletion (FIG. 1). A. tumefaciens EHA105 was selected as it is a highly virulent strain that does not cause tumors (i.e., a “disarmed” strain; Hood et al., 1993).

A 7.2 kb XhoI fragment containing the entire VirD operon from pE702 (pEHC13) was cloned into the XhoI site of pBluescript ks⁺ to make pE3332. A 3.27 kb SphI-klenow blunt-ended XhoI fragment was thereafter cloned from pE3332 into the SmaI-XhoI site of pE3351 (an Asp718 site filled in from pBluescript ks+) to result in pE3353. Thereafter, a 914 base pair (bp) HindIII fragment from pE3052 (internal fragment of an octopine VirD2 gene) was cloned into a second HindIII fragment removed from pE3353 to result in pE3355.

A 885 bp KpnI fragment (internal of HindIII sites) was then deleted from pE3355 to make pE3356, and a XhoI-NotI fragment (containing the P_{virD}-VirD1 partial VirD2 and VirD4 region) from pE3356 was moved onto pJQ200sk (a suicide plasmid, pE1416) to make pE3358.

This VirD2 deletion operon (on pE3358) was then recombined with the disarmed pTiBo542 plasmid (pTiEHA105) in Agrobacterium using sacB as a counter-selection.

### Example 2

### Mutant VirD2 Synthesis

After generating the VirD2-deleted EHA105 Agrobacterium strain as the host for the various mutant VirD2 genes (Example 1), focus was turned to mutagenizing VirD2. An EcoRI-SphI fragment containing P_{virD}-VirD1-DIrD2 was cloned into these same sites of pE4533, a plasmid modified from a binary vector, containing a pVS origin of replication and a spectinomycin-resistance gene. The VirD2 region of this plasmid was polymerase chain reaction (PCR) mutagenized using expired OneTaq DNA polymerase (OneTaq® DNA Polymerase—New England Biolabs®) and the Primers 195+196, and Primers 185+186 listed in Table 1.

TABLE 1

| Primer | |
|---|---|
| Set | Sequences |
| Primer | AATTTCTAGACTTGACCACGCACCTGACG (SEQ ID NO: 6) |
| 195 + | ATACGCGGATCCGTGCGTCGGC (SEQ ID NO: 7) |
| Primer | |
| 196 | |
| Primer | TTAAGTTGGGTAACGCCAGGG (SEQ ID NO: 8) |
| 185 + | AACAACCGCTTGAACAGCAC (SEQ ID NO: 9) |
| Primer | |
| 186 | |
| Primer | GAAGTCTCAATCCCGAAATGC (SEQ ID NO: 11) |
| 683 + | ATTGCATGCATTGGACAGTGCAGAGCT (SEQ ID NO: 33) |
| Primer | |
| 684 | |
| Primer | TTAATCTAGATTTATCTTCTACAAGGAGGTCCCATGGCACCCGATCGCGC |
| 866 | TCAAGTAATC (SEQ ID NO: 34) |

Primer 195 combined with Primer 196 to amplify/mutagenize the N-terminal region of VirD2 (later cut with XbaI and BamHI for ligation into the appropriate vector). Primer 185 and Primer 186 were used to amplify/mutagenize the C-terminal region of VirD2 (later cut with BamHI and SphI for ligation into the appropriate vector). Primers 683+684 were used for sequencing of the VirD2 mutants (with Primer 683 for sequencing VirD2 mutants (from near the BamHI site in the upstream direction) and Primer 684 for sequencing VirD2 mutants (coming from the 3′ end)). Primer 186 was also used for sequencing from upstream of the BamHI site going towards the 3′ end of VirD2.

In some constructions, an enhanced Shine-Dalgarno (SD) ribosome binding sequence was added in front of the VirD2 gene to enhance translation of the mutagenized VirD2 proteins in Agrobacterium. For example, an enhanced Agrobacterium SD sequence having SEQ ID NO: 31 was added in pE4896 and all plasmids derived therefrom.

Further Primers 866 and 683 were used to create a PCR fragment (XbaI-SD-NcoI-N-terminal VirD2-BamHI) to add in a SD sequence on pE4700 (containing a VirD2 gene with little extra 3′ flanking sequence).

The sequences of the wild-type VirD2 and certain VirD2 mutants hereof are shown in FIGS. 2A-2F.

### Example 3

### Transformation Assays

Agrobacterium strains containing mutant VirD2 alleles were first tested on Kalanchoe diagremontiana leaves for stable transformation, using a tumorigenic Agrobacterium strain lacking VirD2. The leaf was scored with a toothpick and the various mutant Agrobacterium strains were individually inoculated into the wounds. The leaves were visualized for tumor formation and photographed one month later (FIG. 6).

The positive control showed tumors, and the negative control showed a wound response. The mutants that did not cause tumors (lack of stable transformation) were advanced for further analysis, which included At2194(5F09) and At2194 (4E12; VirD2 Mutant 2).

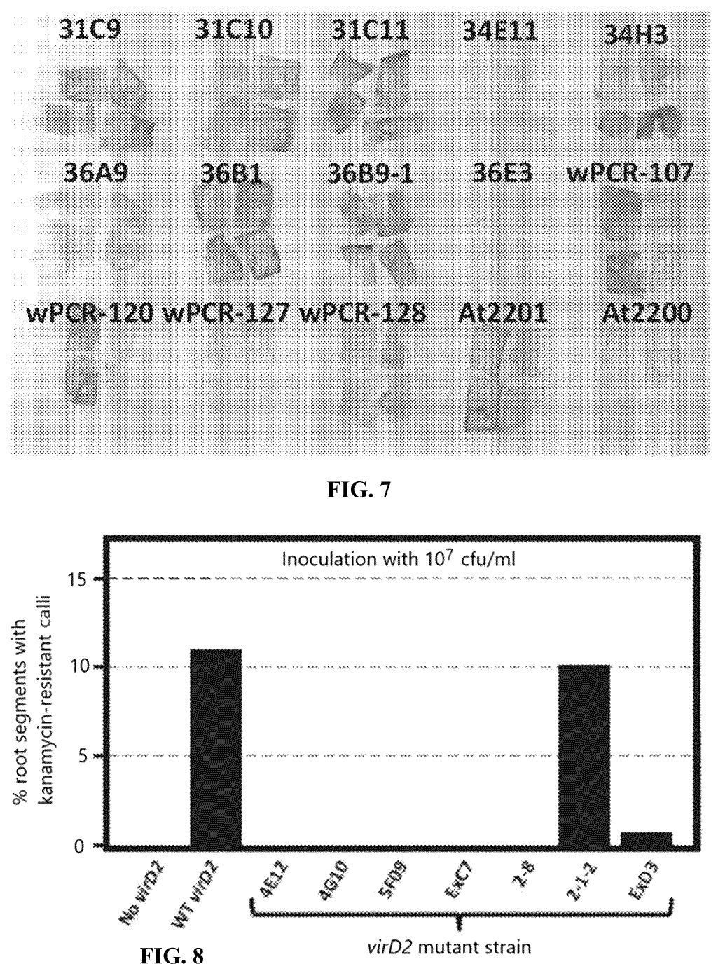

The advanced mutants were then tested for transient transformation using a tobacco leaf infiltration assay. A T-DNA binary vector containing a plant-active gusA-intron gene (pBISN1) was introduced into each of the Agrobacterium VirD2 mutant strains and used to infiltrate Nicotiana benthamiana (tobacco) leaves. After various numbers of days, the infiltrated areas were stained with X-gluc, then destained with 70% alcohol, to visualize GUS activity (a measurement of transient transformation; FIG. 11). The mutants that stimulated production of GUS activity were advanced for further analysis.

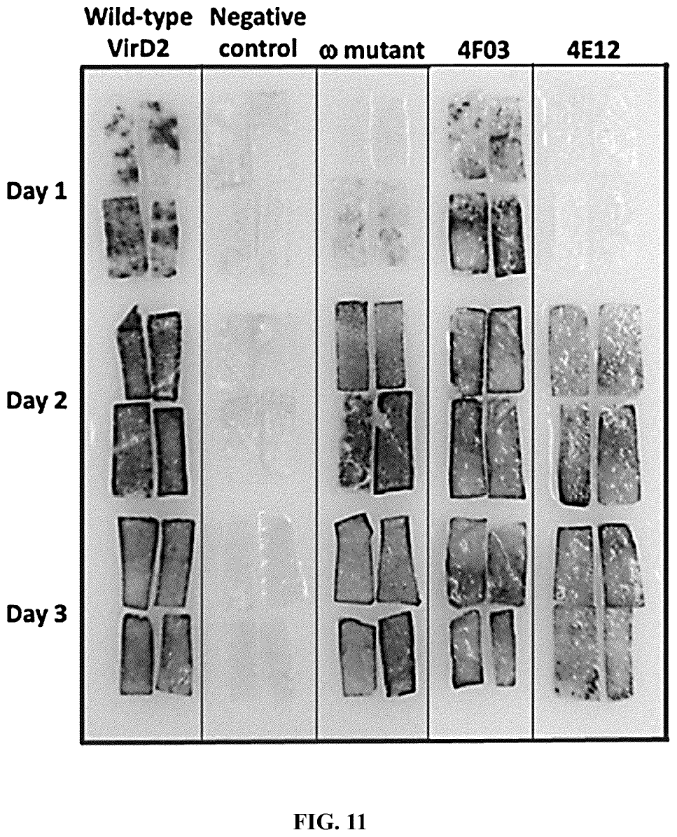

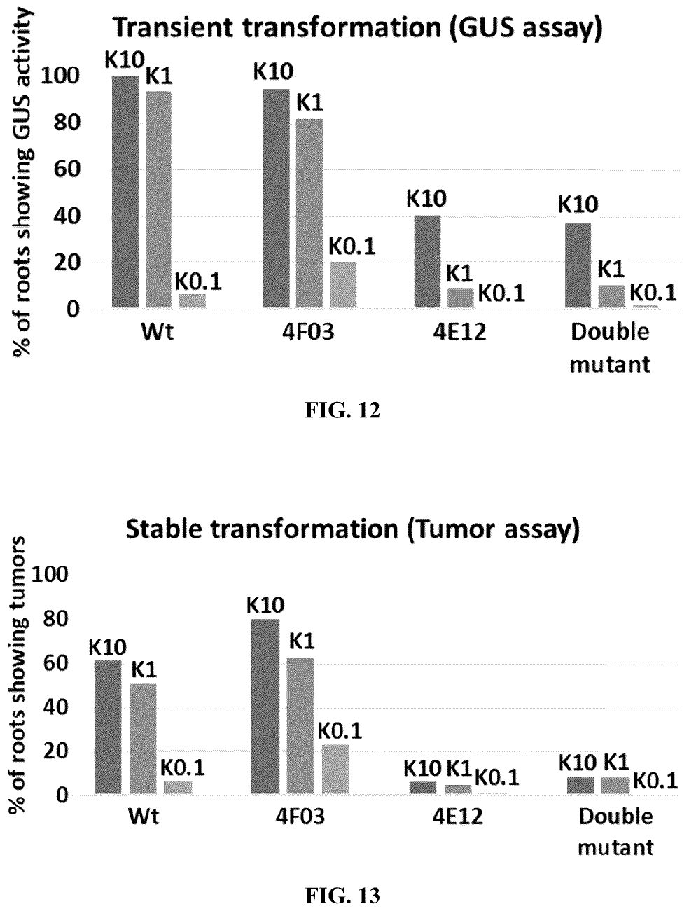

The advanced mutants were then tested using a quantitative Arabidopsis root segment transformation assay described in Gelvin, Agrobacterium transformation of Arabidopsis thaliana roots: A quantitative assay, In Methods in Molecular Biology: Agrobacterium protocols (44): 105-113, Wang, ed., Humana Press (2006). Mutant 4F03 (VirD2 Mutant 1) was hyper-virulent as compared to the wild-type VirD2 gene. Other mutants stimulated very low stable transformation activity, but moderate levels of transient transformation activity (Table 2 and FIGS. 12-15).

TABLE 2
Listed first amino acid and number, identity and position
in the wild-type protein; second amino acid, new
amino acid at this position in the mutant.

| Strain ID | Amino acid change in VirD2 |
|---|---|
| 32A1 | Wild-type |
| 31F6 | Gly³⁶⁷Asp |
| 31G7 | Ser³²⁰Pro |
| 4F03 | Thr³¹⁹Ala |
| ωPCR-11 | Ala³⁵⁸Ser |
| ωPCR-16 | Lys³⁵³Glu |
| ωPCR-25 | Val²⁷⁷Ala |
| ωPCR-39 | Thr³⁸⁸Ser |
| ωPCR-45 | Ser³⁶²Pro |
| ωPCR-55 | Lys³³⁸Glu |

### Example 4

### Mutagenization Assays

Agrobacterium strains harboring either a wild-type VirD2 gene (Wt) or the mutated VirD2 genes 4F03 (VirD2 Mutant 1), 4E12 (VirD2 Mutant 2), or double mutant 4F03+4E12 (VirD2 Mutant 3) were then evaluated for their ability to genome edit (mutagenize) the PDS2 gene of N. benthamiana. For these assays, the VirD2 deletion mutant Agrobacterium strain EHA105 that also contained a T-DNA binary vector encoding Cas9 and a single guide RNA targeting PDS2 was used together with a replicating plasmid that encoded either the wild-type or various mutant VirD2 genes listed above. The sequence of the guide RNA protospacer targeting N. benthamiana PDS2 gene was that of SEQ ID NO: 12.

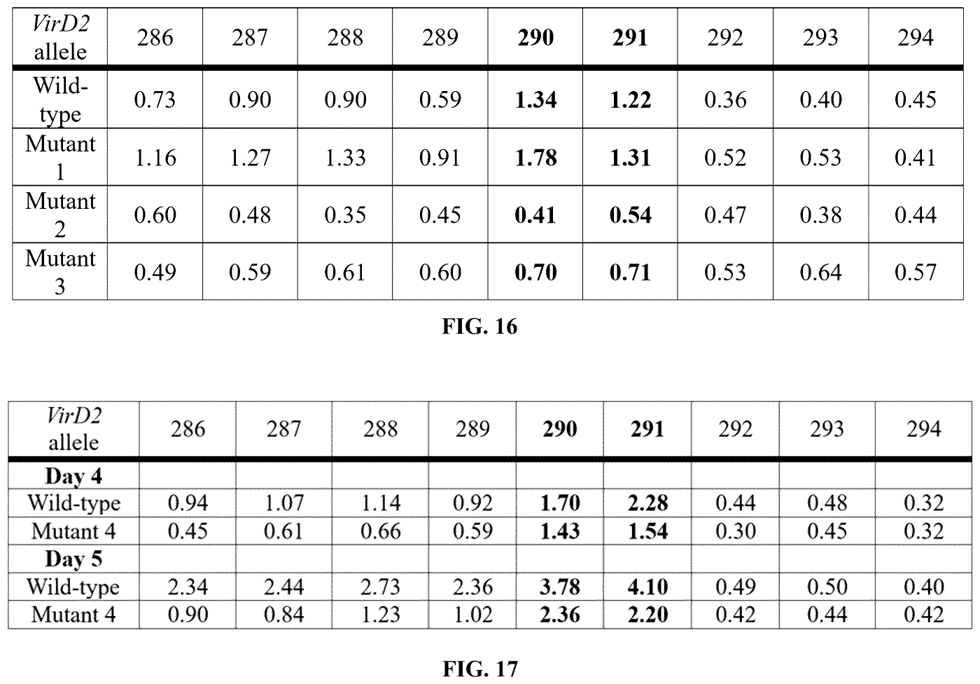

Four to six days after infiltration of the N. benthamiana leaves with various strains, tobacco DNA was isolated from the infiltrated region, the PDS2 gene was amplified using PCR, and the resulting amplicons were sequenced using Wide-seq analysis. The percentage of mutations that appeared in the DNA sequence surrounding the Cas9 cleavage site was then calculated. The results of these experiments are shown in FIGS. 16 and 17.

The F403 mutant (VirD2 Mutant 1) protein edited to about a 10%-30% greater extent than did the wild-type VirD2. The 4E12 mutant (VirD2 Mutant 2) edited at about a 30%-45% lower extent as compared to the wild-type VirD2, but combining this mutation with the 4F03 mutation (VirD2 Mutant 3) mediated the mutation extent to about 55%-70% that of the wild-type VirD2.

### Example 5

### Generating Mutant Agrobacterium Strains and Expression Constructs

The mutant Agrobacterium strains built contained a Virulence helper plasmid (containing the Vir genes) with most of the wild-type VirD2 therein deleted. The mutant VirD2 genes were introduced into these bacterial cells on a separate plasmid and it was confirmed the VirD2 mutant Agrobacterium strains containing the mutant VirD2 genes easily incorporated into the Vir helper plasmid.

The newly constructed vector was comprised of the Vir helper plasmid pTiEHA105, a disarmed (non-tumorigenic) hypervirulent plasmid frequently used to generate transgenic plants, with a mutant VirD2 gene (FIGS. 2A-2F) operably incorporated into the helper plasmid. Similar constructs were also made by operably inserting the mutant VirD2 nucleic acid sequences into other suitable Ti- and Ri-plasmids.

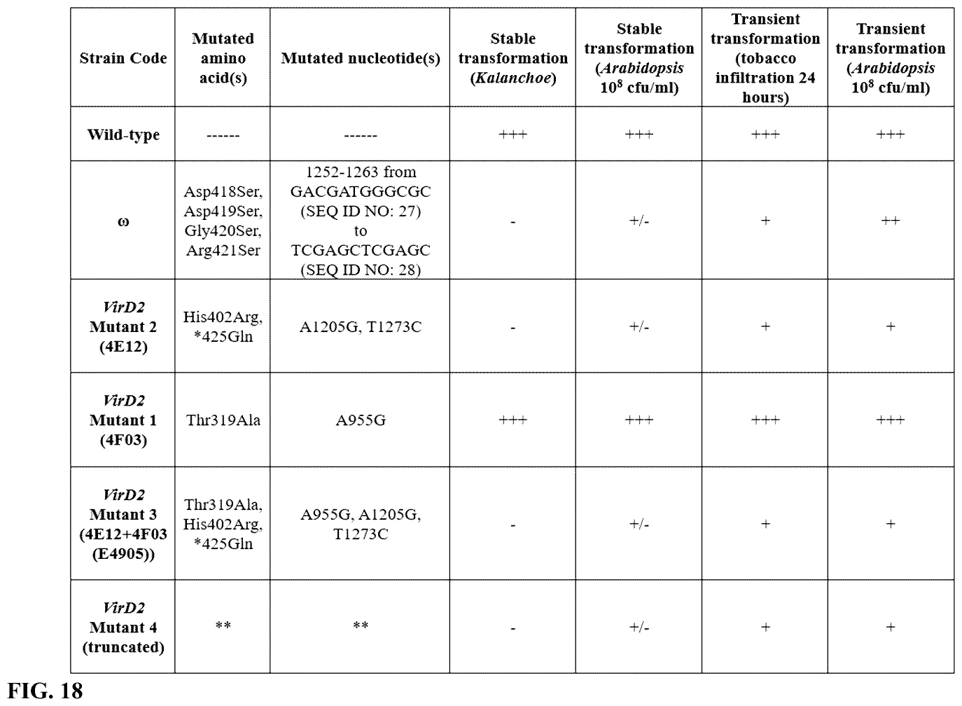

The protocol for making such constructs is similar to that described in Lee et al., Novel constructions to enable the integration of genes into the Agrobacterium tumefaciens C58 chromosome, Molecular Plant-Microbe Interactions 14: 577-579 (2001). The first step was to clone the mutant VirD2 gene, with at least 1 kbp of flanking sequences, into the suicide plasmid pJK200sk. Quandt & Hynes, Versatile suicide vectors which allow direct selection for gene replacement in Gram-negative bacteria, Gene 127: 15-21 (1993). This plasmid was then introduced into A. tumefaciens EHA105 by electroporation and selection was performed for gentamicin resistance. Screening was done for single colonies by growth on medium lacking gentamicin, but containing sucrose (sacB sucrose selection). Colonies that survived this selection regime were characterized by PCR to verify the exchange of the mutant VirD2 gene into pTiEHA105, or other Ti- or Ri-plasmids, and replacement of the wild-type VirD2 gene with the mutant VirD2 gene (FIG. 18).

### Example 6

### Transformation Assays of Tr Mutant (VirD2 Mutant 4)

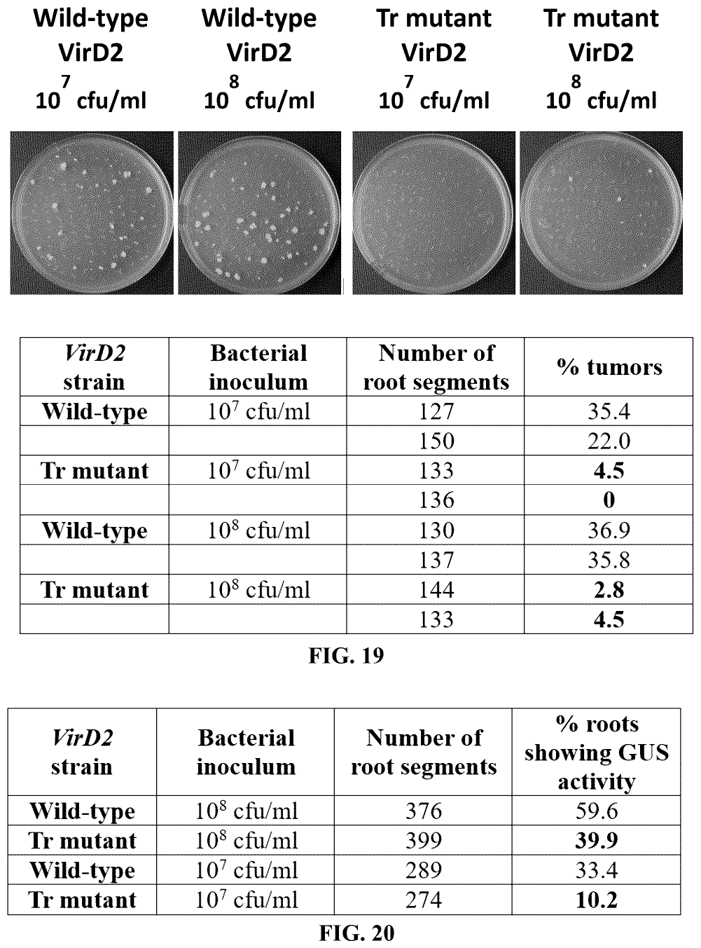

For stable transformation studies, root segments of Arabidopsis (ecotype Col-0) were infected with at either 10⁷ cfu/ml or 10⁸ cfu/ml dosages of a wild-type or Tr mutant (VirD2 Mutant 4) Agrobacterium strains. Both wild-type and the Tr mutant test groups further included oncogenes on the T-DNA. The Tr mutant was prepared pursuant to the herein described protocols. Tumors were scored 1 month post inoculation (FIG. 19).

For transient transformation studies, root segments of Arabidopsis (ecotype Col-0) were infected with at either 10⁷ cfu/ml or 10⁸ cfu/ml dosages of either A. tumefaciens EHA105 or the Tr mutant EHA105 Agrobacterium strain containing the mutation on the Ti-plasmid. The strains also harbored the T-DNA binary vector pBISN1 containing a gusA-intron gene for GUS analysis.

After 6 days, the root segments were stained for GUS activity using X-gluc (FIG. 20). The Tr mutant strain transiently transformed the root segments about 31%-67% as well as did the wild-type VirD2 strain.

### Example 7

### CRISPR Mutagenesis (VirD2 Mutant 4)

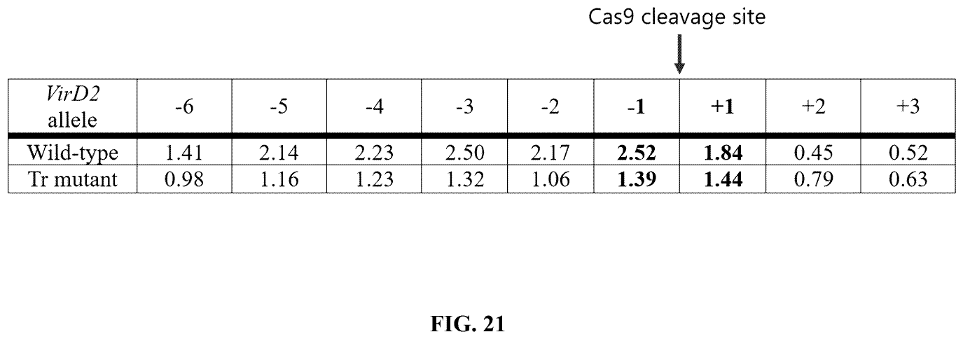

CRISPR mutagenesis of the N. benthamiana PDS2 gene was assessed using Agrobacterium strains harboring either a wild-type (control) or a Tr mutant (VirD2 Mutant 4) VirD2 gene on the Ti-plasmid of strain EHA105. Tobacco leaves were individually infiltrated by the two strains and, 8 days post inoculation, the DNA was extracted using protocols commonly known in the art. The PDS2 gene was amplified by PCR and the amplicons were deep-sequenced using Wide-seq (FIG. 21). The Tr mutant Agrobacterium strain (VirD2 Mutant 4) effected CRISPR mutagenesis about 55%-78% as well as did the wild-type strain in the nucleotides immediately surrounding the Cas9 cleavage site.

1. An Agrobacterium strain comprising a mutant VirD2 gene that effects transient transformation of a plant, the mutant VirD2 strain encoding a wild-type VirD2 protein except for:
(i) a mutation at a threonine at position 319 of the VirD2 protein;
(ii) a mutation at a threonine at position 319 of the VirD2 protein, a mutation at a histidine at position 402 of the VirD2 protein, and an insertion of 30 to 40 amino acid residues at a terminal end of the VirD2 protein resulting from a truncation mutation at a glutamine at position 425; or
(iii) an insertion of 1 to 20 amino acid residues after position 326 of the VirD2 protein, which affects a downstream frameshift.

2. The Agrobacterium strain of claim 1, wherein the wild-type VirD2 protein is or comprises SEQ ID NO: 16 or SEQ ID NO: 17.

3. The Agrobacterium strain of claim 1, wherein the mutant VirD2 gene is preceded by an enhanced Shine-Dalgarno sequence such as SEQ ID NO: 31 or a functional variant of SEQ ID NO: 31.

4. The Agrobacterium strain of claim 1, wherein:
the mutation at position 319 of the VirD2 protein comprises substitution of threonine with an amino acid other than threonine;
the mutation at position 402 of the VirD2 protein comprises substitution of histidine with an amino acid other than histidine; and/or
the truncation of position 425 of the VirD2 protein results from a mutation of a final stop codon encoded by a wild-type VirD2 gene.

5. The Agrobacterium strain of claim 1, wherein the insertion of 1 to 20 amino acid residues after position 326 of the VirD2 protein is or comprises SEQ ID NO: 30 or a functional variant thereof.

6. The Agrobacterium strain of claim 1, wherein the insertion of 30 to 40 amino acid residues is or comprises SEQ ID NO: 32 or a functional variant thereof.

7. The Agrobacterium strain of claim 1, wherein the VirD2 protein is or comprises SEQ ID NO: 19, SEQ ID NO: 23, SEQ ID NO: 25, or a functional variant of SEQ ID NO: 19, SEQ ID NO: 23, or SEQ ID NO: 25.

8. The Agrobacterium strain of claim 1, wherein the mutant VirD2 gene is or comprises SEQ ID NO: 20, SEQ ID NO: 24, SEQ ID NO: 26, or a functional variant of SEQ ID NO: 20, SEQ ID NO: 24, or SEQ ID NO: 26.

9. An expression construct comprising a mutant VirD2 gene that encodes a wild-type Agrobacterium VirD2 protein except for:
(i) a mutation at a threonine at position 319 of the VirD2 protein;
(ii) a mutation at a threonine at position 319 of the VirD2 protein, a mutation at a histidine at position 402 of the VirD2 protein, and an insertion of 30 to 40 amino acid residues at a terminal end of the VirD2 protein due to a truncation mutation at a glutamine at position 425; or
(iii) an insertion of 1 to 22 amino acid residues after position 326 of the VirD2 protein, which affects a downstream frameshift.

10. The expression construct of claim 9, comprising a nucleic acid construct such as a plasmid or a replicating plasmid.

11. The expression construct of claim 9, wherein the expression construct comprises a root-inducing plasmid (Ri-plasmid) or a tumor-inducing plasmid (Ti-plasmid).

12. The expression construct of claim 11, wherein the Ti-plasmid is a pTiEHA105 plasmid.

13. The expression construct of claim 9, wherein a wild-type VirD2 gene of the expression construct is inactive, deleted, disrupted, and/or replaced by the mutant VirD2 gene.

14. The expression construct of claim 9, wherein the mutant VirD2 gene is preceded by an enhanced Shine-Dalgarno sequence such as SEQ ID NO: 31 or a functional variant of SEQ ID NO: 31.

15. The expression construct of claim 9, wherein:
the VirD2 protein is or comprises SEQ ID NO: 19, SEQ ID NO: 23, SEQ ID NO: 25, or a functional variant of SEQ ID NO: 19, SEQ ID NO: 23, or SEQ ID NO: 25; or
the mutant VirD2 gene is or comprises SEQ ID NO: 20, SEQ ID NO: 24, SEQ ID NO: 26, or a functional variant of SEQ ID NO: 20, SEQ ID NO: 24, or SEQ ID NO: 26.

16. The expression construct of claim 9, further comprising one or more operating regulatory segments.

17. The expression construct of claim 9, further comprising a sequence-specific nuclease, a DNA polymerase, and/or a DNA polymerase recruitment protein, or an expression construct comprising a nucleotide sequence encoding the sequence-specific nuclease, the DNA polymerase and/or the DNA polymerase recruitment protein.

18. A method for Agrobacterium-mediated incorporation of exogenous expressible nucleic acids into a host plant material, the method comprising infecting a target host plant material with the Agrobacterium strain comprising a mutant VirD2 gene that effects transient transformation of a plant, the mutant VirD2 strain encoding a wild-type VirD2 protein except for:
(i) a mutation at a threonine at position 319 of the VirD2 protein;
(ii) a mutation at a threonine at position 319 of the VirD2 protein, a mutation at a histidine at position 402 of the VirD2 protein, and an insertion of 30 to 40 amino acid residues at a terminal end of the VirD2 protein resulting from a truncation mutation at a glutamine at position 425; or
(iii) an insertion of 1 to 20 amino acid residues after position 326 of the VirD2 protein, which affects a downstream frameshift.

19. The method of claim 18, wherein infecting comprises inoculating the target host plant material with the Agrobacterium strain at a dose of at or between about 10⁶ cfu/ml to about 10⁹ cfu/ml.

20. The method of claim 19, wherein the dose is at or about 10⁸ cfu/ml.


SEQUENCE LISTING (34 sequences)

SEQ ID NO: 1 - protein, 35 aa, synthetic construct
  source 1..35: note = amino acid extension sequence for mutant 4E12 Agrobacterium strain
RQEGPNNGKWSVHDTLCWPGLRRTDGRTPTSRIRL

SEQ ID NO: 2 - protein, 16 aa, synthetic construct
  source 1..16: note = amino acid extension sequence of mutant VirD2 protein from ExD3 mutant Agrobacterium strain
DVMTRRQVRAEQTVKD

SEQ ID NO: 3 - protein, 68 aa, synthetic construct
  source 1..68: note = amino acid extension sequence at terminal end of protein from w mutant Agrobacterium strain
RDRDRIMANGQFTIRSAGPASVGLTGERRGAASASSSASSNACQPPQGSPRDQSTLIQPLRCYSAVGF

SEQ ID NO: 4 - DNA, 21 bp, synthetic construct
  source 1..21: mol_type = other DNA; note = synthetic primer
gaccatgattacgaatcgagc

SEQ ID NO: 5 - DNA, 31 bp, synthetic construct
  source 1..31: mol_type = other DNA; note = synthetic primer
aatttctagagggcacctttcaatagcgagc

SEQ ID NO: 6 - DNA, 29 bp, synthetic construct
  source 1..29: mol_type = other DNA; note = Primer 195
aatttctagacttgaccacgcacctgacg

SEQ ID NO: 7 - DNA, 22 bp, synthetic construct
  source 1..22: mol_type = other DNA; note = Primer 196
atacgcggatccgtgcgtcggc

SEQ ID NO: 8 - DNA, 21 bp, synthetic construct
  source 1..21: mol_type = other DNA; note = Primer 185
ttaagttgggtaacgccaggg

SEQ ID NO: 9 - DNA, 20 bp, synthetic construct
  source 1..20: mol_type = other DNA; note = Primer 186
aacaaccgcttgaacagcac

SEQ ID NO: 10 - DNA, 29 bp, synthetic construct
  source 1..29: mol_type = other DNA; note = synthetic primer
attgcatgcattggacagtgcagagctag

SEQ ID NO: 11 - DNA, 21 bp, synthetic construct
  source 1..21: mol_type = other DNA; note = Primer 683
gaagtctcaatcccgaaatgc

SEQ ID NO: 12 - RNA, 20 nt, synthetic construct
  source 1..20: mol_type = other RNA; note = guide RNA protospacer targeting a PDS2 gene of Nicotiana benthamiana
gctgcatggaaagatgatga

SEQ ID NO: 13 - protein, 29 aa, Agrobacterium sp.
  source 1..29: note = Wild-type VirD2 protein
KRPPDRHDGELGGRLRARGNRRDDGRGGT

SEQ ID NO: 14 - protein, 4 aa, synthetic construct
  source 1..4: note = synthetic peptide
SSSS

SEQ ID NO: 15 - DNA, 1275 bp, Agrobacterium sp.
  source 1..1275: mol_type = genomic DNA; note = Wild-type VirD2 gene
atgcccgatcgcgctcaagtaatcattcgcattgtgccaggaggtggaaccaagacccttcagcagataatcaatcagttggagtacctgtcccgtaagggaaagctggaactgcagcgttcagcccggcatctcgatattcccgttccgccggatcaaatccgtgagcttgcccaaagctgggttacggaggccgggatttatgacgaaagtcagtcagacgatgataggcaacaagacttaacaacacacattattgtaagcttccccgcaggtaccgaccaaaccgcagcttatgaagccagccgggaatgggcagccgagatgtttgggtcaggatacgggggtggccgctataactatctgacagcctaccacgtcgaccgcgatcatccacatttacatgtcgtggtcaatcgtcgggaacttctggggcacgggtggctgaaaatatccaggcgccatccccagctgaattatgacggcttacggaaaaagatggcagagatttcacttcgtcacggcatagtcctggatgcgacttcgcgagcagaaaggggaatagcagagcgaccaatcacatatgctgaacatcgccgccttgagcggatgcaggctcaaaagattcaattcgaagatacagattttgatgagacctcgcctgaggaagatcgtcgggacctcagtcaatcgttcgatccatttcgatcggacccatctaccggcgaaccggaccgtgcaacccgacatgacaaacaaccgcttgaacagcacgcccgtttccaggagtccgccggctccagcatcaaagccgacgcacggatccgcgtatcattggagagcgagcggagtgcccaaccatccgcgtccaaaatccctgtaattgggcatttcgggattgagacttcctatgtcgctgaagccagcgtgcgcaaacgaagcggcattttcggtacttctcgcccggtgactgacgttgccatgcacacagtcaagcgccagcagcgatcaaaacgacgtaatgacgaggaggcaggtccgagcggagcaaaccgtaaaggattgaaggctgcgcaagttgattccgaggcaaatgtcggtgagcaagacactcgcgatgacagcaacaaggcggctgatccggtgtctgcttccatcggtaccgagcaaccggaagcttctccaaagcgtccgcgtgaccgtcacgatggagaattgggtggacgcaaacgtgcaagaggtaatcgtcgcgacgatgggcgcggggggacctag

SEQ ID NO: 16 - protein, 424 aa, Agrobacterium sp.
  source 1..424: note = Wild-type VirD2 protein
MPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQAQKIQFEDTDFDETSPEEDRRDLSQSFDPFRSDPSTGEPDRATRHDKQPLEQHARFQESAGSSIKADARIRVSLESERSAQPSASKIPVIGHFGIETSYVAEASVRKRSGIFGTSRPVTDVAMHTVKRQQRSKRRNDEEAGPSGANRKGLKAAQVDSEANVGEQDTRDDSNKAADPVSASIGTEQPEASPKRPRDRHDGELGGRKRARGNRRDDGRGGT

SEQ ID NO: 17 - protein, 425 aa, synthetic construct
  source 1..425: note = Wild-type Agrobacterium VirD2 protein modified to include Shine-Dalgarno sequence and NcoI site
MAPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQAQKIQFEDTDFDETSPEEDRRDLSQSFDPFRSDPSTGEPDRATRHDKQPLEQHARFQESAGSSIKADARIRVSLESERSAQPSASKIPVIGHFGIETSYVAEASVRKRSGIFGTSRPVTDVAMHTVKRQQRSKRRNDEEAGPSGANRKGLKAAQVDSEANVGEQDTRDDSNKAADPVSASIGTEQPEASPKRPRDRHDGELGGRKRARGNRRDDGRGGT

SEQ ID NO: 18 - DNA, 1278 bp, synthetic construct
  source 1..1278: mol_type = other DNA; note = Wild-type Agrobacterium VirD2 gene modified to include Shine-Dalgarno sequence and NcoI site
atggcacccgatcgcgctcaagtaatcattcgcattgtgccaggaggtggaaccaagacccttcagcagataatcaatcagttggagtacctgtcccgtaagggaaagctggaactgcagcgttcagcccggcatctcgatattcccgttccgccggatcaaatccgtgagcttgcccaaagctgggttacggaggccgggatttatgacgaaagtcagtcagacgatgataggcaacaagacttaacaacacacattattgtaagcttccccgcaggtaccgaccaaaccgcagcttatgaagccagccgggaatgggcagccgagatgtttgggtcaggatacgggggtggccgctataactatctgacagcctaccacgtcgaccgcgatcatccacatttacatgtcgtggtcaatcgtcgggaacttctggggcacgggtggctgaaaatatccaggcgccatccccagctgaattatgacggcttacggaaaaagatggcagagatttcacttcgtcacggcatagtcctggatgcgacttcgcgagcagaaaggggaatagcagagcgaccaatcacatatgctgaacatcgccgccttgagcggatgcaggctcaaaagattcaattcgaagatacagattttgatgagacctcgcctgaggaagatcgtcgggacctcagtcaatcgttcgatccatttcgatcggacccatctaccggcgaaccggaccgtgcaacccgacatgacaaacaaccgcttgaacagcacgcccgtttccaggagtccgccggctccagcatcaaagccgacgcacggatccgcgtatcattggagagcgagcggagtgcccaaccatccgcgtccaaaatccctgtaattgggcatttcgggattgagacttcctatgtcgctgaagccagcgtgcgcaaacgaagcggcattttcggtacttctcgcccggtgactgacgttgccatgcacacagtcaagcgccagcagcgatcaaaacgacgtaatgacgaggaggcaggtccgagcggagcaaaccgtaaaggattgaaggctgcgcaagttgattccgaggcaaatgtcggtgagcaagacactcgcgatgacagcaacaaggcggctgatccggtgtctgcttccatcggtaccgagcaaccggaagcttctccaaagcgtccgcgtgaccgtcacgatggagaattgggtggacgcaaacgtgcaagaggtaatcgtcgcgacgatgggcgcggggggacctag

SEQ ID NO: 19 - protein, 425 aa, synthetic construct
  source 1..425: note = VirD2 mutant #1
MAPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQAQKIQFEDTDFDETSPEEDRRDLSQSFDPFRSDPSTGEPDRATRHDKQPLEQHARFQESAGSSIKADARIRVSLESERSAQPSASKIPVIGHFGIETSYVAEASVRKRSGIFGASRPVTDVAMHTVKRQQRSKRRNDEEAGPSGANRKGLKAAQVDSEANVGEQDTRDDSNKAADPVSASIGTEQPEASPKRPRDRHDGELGGRKRARGNRRDDGRGGT

SEQ ID NO: 20 - DNA, 1278 bp, synthetic construct; identical to SEQ ID NO: 18
  source 1..1278: mol_type = other DNA; note = VirD2 mutant #1

SEQ ID NO: 21 - protein, 461 aa, synthetic construct
  source 1..461: note = VirD2 mutant #2
MAPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQAQKIQFEDTDFDETSPEEDRRDLSQSFDPFRSDPSTGEPDRATRHDKQPLEQHARFQESAGSSIKADARIRVSLESERSAQPSASKIPVIGHFGIETSYVAEASVRKRSGIFGTSRPVTDVAMHTVKRQQRSKRRNDEEAGPSGANRKGLKAAQVDSEANVGEQDTRDDSNKAADPVSASIGTEQPEASPKRPRDRRDGELGGRKRARGNRRDDGRGGTQRQEGPNNGKWSVHDTLCWPGLRRTDGRTPWSRIRL

SEQ ID NO: 22 - DNA, 1386 bp, synthetic construct
  source 1..1386: mol_type = other DNA; note = VirD2 mutant #2
atggcacccgatcgcgctcaagtaatcattcgcattgtgccaggaggtggaaccaagacccttcagcagataatcaatcagttggagtacctgtcccgtaagggaaagctggaactgcagcgttcagcccggcatctcgatattcccgttccgccggatcaaatccgtgagcttgcccaaagctgggttacggaggccgggatttatgacgaaagtcagtcagacgatgataggcaacaagacttaacaacacacattattgtaagcttccccgcaggtaccgaccaaaccgcagcttatgaagccagccgggaatgggcagccgagatgtttgggtcaggatacgggggtggccgctataactatctgacagcctaccacgtcgaccgcgatcatccacatttacatgtcgtggtcaatcgtcgggaacttctggggcacgggtggctgaaaatatccaggcgccatccccagctgaattatgacggcttacggaaaaagatggcagagatttcacttcgtcacggcatagtcctggatgcgacttcgcgagcagaaaggggaatagcagagcgaccaatcacatatgctgaacatcgccgccttgagcggatgcaggctcaaaagattcaattcgaagatacagattttgatgagacctcgcctgaggaagatcgtcgggacctcagtcaatcgttcgatccatttcgatcggacccatctaccggcgaaccggaccgtgcaacccgacatgacaaacaaccgcttgaacagcacgcccgtttccaggagtccgccggctccagcatcaaagccgacgcacggatccgcgtatcattggagagcgagcggagtgcccaaccatccgcgtccaaaatccctgtaattgggcatttcgggattgagacttcctatgtcgctgaagccagcgtgcgcaaacgaagcggcattttcggtacttctcgcccggtgactgacgttgccatgcacacagtcaagcgccagcagcgatcaaaacgacgtaatgacgaggaggcaggtccgagcggagcaaaccgtaaaggattgaaggctgcgcaagttgattccgaggcaaatgtcggtgagcaagacactcgcgatgacagcaacaaggcggctgatccggtgtctgcttccatcggtaccgagcaaccggaagcttctccaaagcgtccgcgtgaccgtcgcgatggagaattgggtggacgcaaacgtgcaagaggtaatcgtcgcgacgatgggcgcggggggacccagagacaggaaggaccgaataatggcaaatggtcagttcacgatacgctctgctggcccggcctccgtcggactgacgggcgaacgccgtggagccgcatccgcctctag

SEQ ID NO: 23 - protein, 461 aa, synthetic construct
  source 1..461: note = VirD2 mutant #3
MAPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQAQKIQFEDTDFDETSPEEDRRDLSQSFDPFRSDPSTGEPDRATRHDKQPLEQHARFQESAGSSIKADARIRVSLESERSAQPSASKIPVIGHFGIETSYVAEASVRKRSGIFGASRPVTDVAMHTVKRQQRSKRRNDEEAGPSGANRKGLKAAQVDSEANVGEQDTRDDSNKAADPVSASIGTEQPEASPKRPRDRRDGELGGRKRARGNRRDDGRGGTQRQEGPNNGKWSVHDTLCWPGLRRTDGRTPWSRIRL

SEQ ID NO: 24 - DNA, 1386 bp, synthetic construct
  source 1..1386: mol_type = other DNA; note = VirD2 mutant #3
atggcacccgatcgcgctcaagtaatcattcgcattgtgccaggaggtggaaccaagacccttcagcagataatcaatcagttggagtacctgtcccgtaagggaaagctggaactgcagcgttcagcccggcatctcgatattcccgttccgccggatcaaatccgtgagcttgcccaaagctgggttacggaggccgggatttatgacgaaagtcagtcagacgatgataggcaacaagacttaacaacacacattattgtaagcttccccgcaggtaccgaccaaaccgcagcttatgaagccagccgggaatgggcagccgagatgtttgggtcaggatacgggggtggccgctataactatctgacagcctaccacgtcgaccgcgatcatccacatttacatgtcgtggtcaatcgtcgggaacttctggggcacgggtggctgaaaatatccaggcgccatccccagctgaattatgacggcttacggaaaaagatggcagagatttcacttcgtcacggcatagtcctggatgcgacttcgcgagcagaaaggggaatagcagagcgaccaatcacatatgctgaacatcgccgccttgagcggatgcaggctcaaaagattcaattcgaagatacagattttgatgagacctcgcctgaggaagatcgtcgggacctcagtcaatcgttcgatccatttcgatcggacccatctaccggcgaaccggaccgtgcaacccgacatgacaaacaaccgcttgaacagcacgcccgtttccaggagtccgccggctccagcatcaaagccgacgcacggatccgcgtatcattggagagcgagcggagtgcccaaccatccgcgtccaaaatccctgtaattgggcatttcgggattgagacttcctatgtcgctgaagccagcgtgcgcaaacgaagcggcattttcggtgcttctcgcccggtgactgacgttgccatgcacacagtcaagcgccagcagcgatcaaaacgacgtaatgacgaggaggcaggtccgagcggagcaaaccgtaaaggattgaaggctgcgcaagttgattccgaggcaaatgtcggtgagcaagacactcgcgatgacagcaacaaggcggctgatccggtgtctgcttccatcggtaccgagcaaccggaagcttctccaaagcgtccgcgtgaccgtcgcgatggagaattgggtggacgcaaacgtgcaagaggtaatcgtcgcgacgatgggcgcggggggacccagagacaggaaggaccgaataatggcaaatggtcagttcacgatacgctctgctggcccggcctccgtcggactgacgggcgaacgccgtggagccgcatccgcctctag

SEQ ID NO: 25 - protein, 344 aa, synthetic construct
  source 1..344: note = VirD2 mutant #4
MAPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQAQKIQFEDTDFDETSPEEDRRDLSQSFDPFRSDPSTGEPDRATRHDKQPLEQHARFQESAGSSIKADARIRVSLESERSAQPSASKIPVIGHFGIETSYVAEASVRKRSGIFGTSRPVTDGSQRAQTKRHFRCFSPGD

SEQ ID NO: 26 - DNA, 1104 bp, synthetic construct
  source 1..1104: mol_type = other DNA; note = VirD2 mutant #4
atggcacccgatcgcgctcaagtaatcattcgcattgtgccaggaggtggaaccaagacccttcagcagataatcaatcagttggagtacctgtcccgtaagggaaagctggaactgcagcgttcagcccggcatctcgatattcccgttccgccggatcaaatccgtgagcttgcccaaagctgggttacggaggccgggatttatgacgaaagtcagtcagacgatgataggcaacaagacttaacaacacacattattgtaagcttccccgcaggtaccgaccaaaccgcagcttatgaagccagccgggaatgggcagccgagatgtttgggtcaggatacgggggtggccgctataactatctgacagcctaccacgtcgaccgcgatcatccacatttacatgtcgtggtcaatcgtcgggaacttctggggcacgggtggctgaaaatatccaggcgccatccccagctgaattatgacggcttacggaaaaagatggcagagatttcacttcgtcacggcatagtcctggatgcgacttcgcgagcagaaaggggaatagcagagcgaccaatcacatatgctgaacatcgccgccttgagcggatgcaggctcaaaagattcaattcgaagatacagattttgatgagacctcgcctgaggaagatcgtcgggacctcagtcaatcgttcgatccatttcgatcggacccatctaccggcgaaccggaccgtgcaacccgacatgacaaacaaccgcttgaacagcacgcccgtttccaggagtccgccggctccagcatcaaagccgacgcacggatccgcgtatcattggagagcgagcggagtgcccaaccatccgcgtccaaaatccctgtaattgggcatttcgggattgagacttcctatgtcgctgaagccagcgtgcgcaaacgaagcggcattttcggtacttctcgcccggtgactgacggaagccagcgtgcgcaaacgaagcggcattttcggtgcttctcgcccggtgactgacgttgccatgcacacagtcagcgtgcgcaaacgaagcggcattttcggtgcttctcgcccggtgactga

SEQ ID NO: 27 - DNA, 12 bp, synthetic construct
  source 1..12: mol_type = other DNA; note = mutated nucleotides of w mutant VirD2 strain
gacgatgggcgc

SEQ ID NO: 28 - DNA, 12 bp, synthetic construct
  source 1..12: mol_type = other DNA; note = mutated nucleotides of w mutant VirD2 strain
tcgagctcgagc

SEQ ID NO: 29 - protein, 18 aa, synthetic construct
  source 1..18: note = 18 amino acid residues inserted at terminal end for truncated mutant #4
GSQRAQTKRHFRCFSPGD

SEQ ID NO: 30 - DNA, 125 bp, synthetic construct
  source 1..125: mol_type = other DNA; note = nucleotides for encoding 18 amino acid residues for insertion after position 326 of the VirD2 protein of mutant #4
gaagccagcgtgcgcaaacgaagcggcattttcggtgcttctcgcccggtgactgacgttgccatgcacacagtcagcgtgcgcaaacgaagcggcattttcggtgcttctcgcccggtgactga

SEQ ID NO: 31 - DNA, 20 bp, synthetic construct
  source 1..20: mol_type = other DNA; note = Shine-Dalgarno sequence
tttatcttctacaaggaggt

SEQ ID NO: 32 - protein, 38 aa, synthetic construct
  source 1..38: note = amino acid extension at 425Gln in VirD2 mutant #2 and mutant #3
QRQEGPNNGKWSVHDTLCWPGLRRTDTDGRTPWSRIRL

SEQ ID NO: 33 - DNA, 27 bp, synthetic construct
  source 1..27: mol_type = other DNA; note = Primer 684
attgcatgcattggacagtgcagagct

SEQ ID NO: 34 - DNA, 60 bp, synthetic construct
  source 1..60: mol_type = other DNA; note = Primer 866
ttaatctagatttatcttctacaaggaggtcccatggcacccgatcgcgctcaagtaatc